(19) United States
(12) Patent Application Publication
(10) Pub. No.: US 2003/0219800 A1
(43) Pub. Date: Nov. 27, 2003

(54) Multiplexed cell transfection using coded carriers

(76) Inventors: Oren E. Beske, Belmont, CA (US); Kimberly Bland, El Cerrito, CA (US); Simon Goldbard, San Jose, CA (US); Michael A. Zarowitz, San Carlos, CA (US)

Correspondence Address: KOLISCH HARTWELL, P.C., 520 S.W. YAMHILL STREET, SUITE 200, PORTLAND, OR, 97204, US

(21) Appl. No.: 10/382,818

(22) Filed: Mar. 5, 2003

Related U.S. Application Data
(63) Continuation-in-part of application No. 10/120,900, filed on Apr. 10, 2002.
(60) Provisional application No. 60/362,238, filed on Mar. 5, 2002.
(60) Provisional application No. 60/413,675, filed on Sep. 24, 2002.

(30) Foreign Application Priority Data
Oct. 18, 2001 PCT/US01/51413

(51) Int. Cl. C12Q 1/68; G01N 33/53; G01N 33/567; C12M 1/34; C12N 15/85

(52) U.S. Cl. 435/6; 435/7.2; 435/455; 435/287.2

(57) ABSTRACT

Systems, including methods, compositions, and kits, for transfection of cells with transfection materials using coded carriers.

### CROSS REFERENCE TO PRIORITY APPLICATIONS

This application is a continuation-in-part of U.S. patent application Ser. No. 10/120,900, filed Apr. 10, 2002. This application also claims the benefit under 35 U.S.C. § 119(e) of the following U.S. provisional patent applications: Serial No. 60/362,238, filed Mar. 5, 2002; and Serial No. 60/413,675, filed Sep. 24, 2002.

U.S. patent application Ser. No. 10/120,900 claims the benefit under 35 U.S.C. § 120 of PCT Application Serial No. PCT/US01/51413, filed Oct. 18, 2001, and published as Publication No. WO 02/37944 on May 16, 2002, which in turn claims priority from the following U.S. provisional patent applications: Serial No. 60/241,714, filed Oct. 18, 2000; Serial No. 60/259,416, filed Dec. 28, 2000; Serial No. 60/293,863, filed May 24, 2001; Serial No. 60/299,267, filed Jun. 18, 2001; Serial No. 60/299,810, filed Jun. 20, 2001; Serial No. 60/307,649, filed Jul. 24, 2001; Serial No. 60/307,650, filed Jul. 24, 2001; Serial No. 60/310,540, filed Aug. 6, 2001; Serial No. 60/317,409, filed Sep. 4, 2001; Serial No. 60/318,156, filed Sep. 7, 2001; and Serial No. 60/328,614, filed Oct. 10, 2001.

The above-identified U.S., PCT, and provisional patent applications are all incorporated herein by reference in their entirety for all purposes.

### CROSS REFERENCES TO RELATED APPLICATIONS

This application incorporates by reference in their entirety for all purposes the following U.S. patent applications: Ser. No. 09/549,970, filed Apr. 14, 2000; Serial No. 09/694,077, filed Oct. 19, 2000; Ser. No. 10/119,814, filed Apr. 9, 2002; Ser. No. 10/186,219, filed Jun. 27, 2002; Ser. No. 10/238,914, filed Sep. 9, 2002; Ser. No. 10/273,605, filed Oct. 18, 2002; Ser. No. 10/282,904, filed Oct. 28, 2002; and Ser. No. 10/282,940, filed Oct. 28, 2002.

This application also incorporates by reference in their entirety for all purposes the following U.S. provisional patent applications: Serial No. 60/362,001, filed Mar. 5, 2002; Serial No. 60/362,055, filed Mar. 5, 2002; Serial No. 60/370,313, filed Apr. 4, 2002; Serial No. 60/383,091, filed May 23, 2002; Serial No. 60/383,092, filed May 23, 2002; Serial No. 60/413,407, filed Sep. 24, 2002; Serial No. 60/421,280, filed Oct. 25, 2002; and Serial No. 60/426,633, filed Nov. 14, 2002.

This application also incorporates by reference in their entirety for all purposes the following PCT patent applications: Serial No. PCT/US00/10181, filed Apr. 14, 2000, and published as Publication No. WO 00/63419 on Oct. 26, 2000; Serial No. PCT/US02/33350, filed Oct. 18, 2002; and Serial No. PCT/US02/34699, filed Oct. 28, 2002.

### CROSS-REFERENCE TO ADDITIONAL MATERIALS

This application also incorporates by reference in their entirety for all purposes the following U.S. Pat. No. 3,772,099, issued Nov. 13, 1973; U.S. Pat. No. 3,897,284, issued Jul. 29, 1975; U.S. Pat. No. 3,964,294, issued Jun. 22, 1976; U.S. Pat. No. 3,966,599, issued Jun. 29, 1976; U.S. Pat. No. 3,980,561, issued Sep. 14, 1976; U.S. Pat. No. 4,053,433, issued Oct. 11, 1977; U.S. Pat. No. 4,087,327, issued May 2, 1978; U.S. Pat. No. 4,131,064, issued Dec. 26, 1978; U.S. Pat. No. 4,197,104, issued Apr. 8, 1980; U.S. Pat. No. 4,329,393, issued May 11, 1982; U.S. Pat. No. 4,343,904, issued Aug. 10, 1982; U.S. Pat. No. 4,363,965, issued Dec. 14, 1982; U.S. Pat. No. 4,390,452, issued Jun. 28, 1983; U.S. Pat. No. 4,469,623, issued Sep. 4, 1984; U.S. Pat. No. 4,634,675, issued Jan. 6, 1987; U.S. Pat. No. 4,640,035, issued Feb. 3, 1987; U.S. Pat. No. 4,649,114, issued Mar. 10, 1987; U.S. Pat. No. 4,652,395, issued Mar. 24, 1987; U.S. Pat. No. 4,727,040, issued Feb. 23, 1988; U.S. Pat. No. 4,833,083, issued May 23, 1989; U.S. Pat. No. 4,888,294, issued Dec. 19, 1989; U.S. Pat. No. 4,906,577, issued Mar. 6, 1990; U.S. Pat. No. 4,921,792, issued May 1, 1990; U.S. No. 4,963,490, issued Oct. 16, 1990; U.S. Pat. No. 4,982,739, issued Jan. 8, 1991; U.S. Pat. No. 5,019,512, issued May 28, 1991; U.S. Pat. No. 5,079,161, issued Jan. 7, 1992; U.S. Pat. No. 5,081,036, issued Jan. 14, 1992; U.S. Pat. No. 5,096,814, issued Mar. 17, 1992; U.S. Pat. No. 5,100,783, issued Mar. 31, 1992; U.S. Pat. No. 5,100,799, issued Mar. 31, 1992; U.S. Pat. No. 5,114,853, issued May 19, 1992; U.S. No. 5,126,269, issued Jun. 30, 1992; U.S. Pat. No. 5,233,369, issued Aug. 3, 1993; U.S. Pat. No. 5,409,839, issued Apr. 25, 1995; U.S. Pat. No. 5,451,505, issued Sep. 19, 1995; U.S. Pat. No. 5,486,855, issued Jan. 23, 1996; U.S. Pat. No. 5,571,410, issued Nov. 5, 1996; U.S. Pat. No. 5,708,153, issued Jan. 13, 1998; U.S. Pat. No. 5,741,462, issued Apr. 21, 1998; U.S. Pat. No. 5,760,394, issued Jun. 2, 1998; U.S. Pat. No. 5,770,455, filed Jun. 23, 1998; U.S. Pat. No. 5,780,258, issued Jul. 14, 1998; issued Jun. 23, 1998; U.S. Pat. No. 5,817,751, issued Oct. 6, 1998; U.S. Pat. No. 5,840,485, issued Nov. 24, 1998; U.S. Pat. No. 5,961,923, issued Oct. 5, 1999; U.S. Pat. No. 5,981,180, issued Nov. 9, 1999; U.S. Pat. No. 5,989,835, issued Nov. 23, 1999; U.S. Pat. No. 5,990,479, issued Nov. 23, 1999; U.S. Pat. No. 6,025,200, issued Feb. 15, 2000; U.S. Pat. No. 6,100,026, issued Aug. 8, 2000; and U.S. Pat. No. 6,103,479, issued Aug. 15, 2000.

This application also incorporates by reference in their entirety for all purposes the following PCT patent applications: Serial No. PCT/IL97/00105, filed Mar. 20, 1997; Serial U.S. Pat. No. PCT/US98/21562, filed Oct. 14, 1998; Serial No. PCT/US98/22785, filed Oct. 27, 1998; Ser. No. PCT/US99/00918, filed Jan. 15, 1999; Ser. No. PCT/US99/01315, filed Jan. 22, 1999; Serial No. PCT/GB99/00457, filed Feb. 15, 1999; Serial No. PCT/US99/14387, filed Jun. 24, 1999; Serial No. PCT/GB99/02108, filed Jul. 2, 1999; Serial No. PCT/SE99/01836, filed Oct. 12, 1999; Serial No. PCT/US99/31022, filed Dec. 28, 1999; Serial No. PCT/US00/25457, filed Sep. 18, 2000; Serial No. PCT/US00/27121, filed Oct. 2, 2000; and Serial No. PCT/US00/41049, filed Oct. 2, 2000.

### FIELD OF THE INVENTION

The invention relates to multiplexed transfection of cells. More particularly, the invention relates to multiplexed transfection of cells using coded carriers carrying transfection materials.

### BACKGROUND

Sequencing the human genome has identified effectively all human genes. However, determining the function of these genes is a much more formidable task, often referred to as functional genomics. To determine the function of a specific gene, the expression of that gene may be altered, and then a physiological parameter may be measured. The choice of which parameter to measure, although strongly influenced by bioinformatics, may be relatively arbitrary. Such parameters may be expression level of a gene or set of genes, activation of a signaling cascade, inhibition of a cell-surface receptor, and/or effect on cell cycle progression, among others.

To alter expression of a gene of interest, the gene may be overexpressed, underexpressed (knocked-down), or gene expression may be abolished completely (knocked-out). For example, introduction of a “sense” expression vector or nucleic acid into cells may be used to effect expression and/or overexpression of a gene of interest. Alternatively, introduction of an antisense expression vector or nucleic acid, or a posttranscriptional gene-silencing (PTGS) agent, such as double-stranded RNA, RNAi, siRNA, etc., may be employed to block expression of a gene of interest. The antisense vector/nucleic acid or PTGS agent should be complementary to and/or overlap with at least a portion of a sense transcript from the gene of interest and generally lowers, or in some cases shuts off, expression of the gene of interest. Although generally effective, each of these approaches requires that a nucleic acid be introduced into cells.

Transfection provides a general approach for introducing nucleic acids into cells. Transfection has been used extensively to introduce sense and antisense nucleic acids, PTGS agents, and expression vectors, as described above, in addition to exogenous reporter genes that encode a readily assayed reporter protein or RNA. Accordingly, cell transfection frequently plays a critical role in identifying new drugs and in analyses of gene/protein function and cell biology.

Molecular biologists have developed a variety of widely used transfection procedures for transfection of adherent cells. Often, these procedures rely on “forward” transfection in which a nucleic acid is presented to cells that are attached to a substrate, such as a cover slip. Appropriate treatment of the nucleic acid and/or the cells with a transfection vehicle such as a lipid promotes uptake of the nucleic acid into the cells.

Despite the prevalence of forward transfection methods, these methods in their current form may not be suited to high-throughput applications involving microarrays. Microarrays generally lack barriers that block fluid movement between samples within each microarray and therefore may not be suited for standard methods of forward transfection. For example, different nucleic acids placed at defined positions in an array on a pre-attached layer of cells would not be restricted to these defined positions, because most cells used for transfection require a constant fluid bath for survival. Thus, efforts to carry out localized addition of a nucleic acid to the cell layer using current forward-transfection methods would be thwarted by the randomizing motion of fluid (and nucleic acid).

By reversing the order in which cells and nucleic acids are placed on a substrate, Sabatini and Collins provide a reverse transfection strategy. This strategy is disclosed in PCT Application No. PCT/US00/25457, filed Sep. 18, 2000, which is incorporated herein by reference. In the Sabatini and Collins strategy, cells are attached to a substrate that has been pre-patterned with an array of nucleic acids. Because the nucleic acid array is pre-patterned, forming the array does not require that fluid bathe the entire surface of the substrate. Therefore, different nucleic acids can be disposed at discrete regions of the substrate, for example, as a printed planar array. Subsequently, cells adhere to the substrate adjacent the printed array of nucleic acids, allowing the nucleic acids to be transfected into a corresponding array pattern of cell populations.

Despite the attractiveness of a fixed, preprinted planar array of nucleic acids for positional transfection, the strategy of Sabatini and Collins has a number of disadvantages, particularly for high-throughput screening. For example, this strategy does not offer a substantial economy of scale when the same array is produced many times. Instead, the amount of printing in forming the arrays remains proportional to the number of arrays produced. Furthermore, the preprinted array may not be modified readily after printing. Thus, it may be difficult to remove or add samples once the array is formed. Due to this lack of flexibility, addition of new candidates or modification of a screening strategy may require preprinted arrays to be discarded and replaced with new arrays. Other disadvantages of a fixed planar array may include increased expense and decreased reproducibility. A planar array may overuse valuable reagents because reagents generally need to be uniformly distributed over the entire array surface, increasing the amount of reagents required. Problems also may arise in ensuring uniform exposure of the entire array to reagents. Furthermore, the planar array may not be compatible with microplates, a significant drawback since microplate compatibility is a standard requirement in the high-throughput environment of modern drug discovery. Therefore, new methods are needed to improve transfection for high-throughput assays.

### SUMMARY OF THE INVENTION

The invention provides systems, including methods, compositions, and kits, for transfection of cells with transfection materials using coded carriers.

### BRIEF DESCRIPTION OF THE DRAWINGS

FIG. 1 is a flowchart of a method for multiplexed analysis of transfected cells using coded carriers to identify the transfected cells, in accordance with aspects of the invention.

FIG. 2 is a schematic view of a method for producing an array of transfected cells by reverse transfection with coded carriers, in accordance with aspects of the invention.

FIG. 3 is a schematic view of a method for screening candidate modulators using the array of FIG. 2, in accordance with aspects of the invention.

FIG. 4 is a schematic view of a method for producing an array of transfected cells by forward transfection with coded carriers, in accordance with aspects of the invention.

FIG. 5 is a plan view of the array of transfected cells from FIG. 4.

FIG. 6 is a schematic sectional view of a coded carrier in apposition to a subset of cells produced by the forward transfection of FIG. 4, in accordance with aspects of the invention.

FIG. 7 is a schematic sectional view of the coded carrier of FIG. 5 in apposition to distinct subsets of cells during combined forward and reverse transfection, in accordance with aspects of the invention.

FIG. 8 is a schematic view of a method for multiplexed analysis of changes in reporter gene activity produced by transfecting antisense nucleic acids using coded carriers, in accordance with aspects of the invention.

FIG. 9 is a graph of dose-response data resulting from (1) transfection of an ecdysone response system into cells using coded carriers, (2) treating the cells with ponasterone (PA), and (3) measuring the different responses, in accordance with aspects of the invention.

FIG. 10 is a graph of dose-response data resulting from (1) transfection of an MC3 response system into cells using coded carriers, (2) treating the cells with different concentrations of MTII, and (3) measuring the different responses, in accordance with aspects of the invention.

FIG. 11 is a bar graph showing selective agonist stimulation of ecdysone and MC3 response systems introduced by multiplexed transfection with coded carriers, in accordance with aspects of the invention.

### DETAILED DESCRIPTION

The invention provides systems, including methods, compositions, and kits, for transfection of cells with transfection materials. These systems involve coded carriers having detectable, distinguishable codes. The transfections use the coded carriers to carry and identify the transfection materials. The coded carriers identify subsets of the cells transfected by the transfection materials through apposition of the cell subsets to the carriers. Accordingly, a plurality of transfections may be conducted in parallel in a single compartment. As a result, the systems described herein may offer significant improvements in flexibility, cost, and reliability over previous transfection and analysis methods. For example, coded carriers may be used to screen and/or analyze libraries of transfection materials and/or the effects of physical modulators and/or libraries of chemical/biological modulators, ligands, and/or antibodies on transfected cells using a multiplexed microplate format.

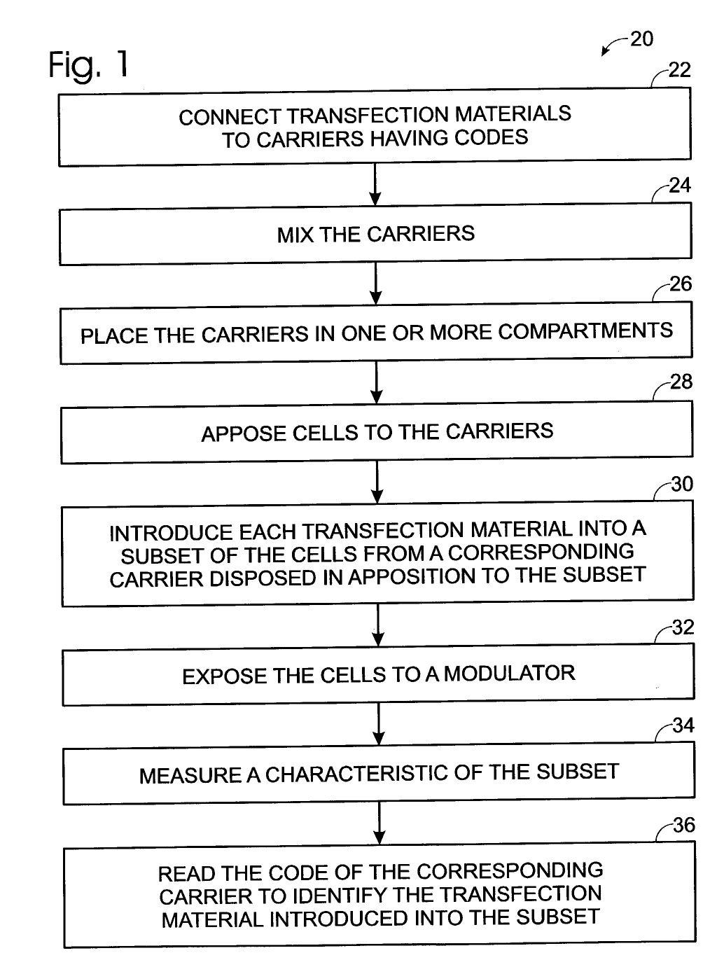

FIG. 1 shows a flowchart of an embodiment of a method 20 for multiplexed analysis of cells using coded carriers to transfect the cells. The method steps shown here may be conducted in any suitable order and/or repeated any suitable number of times. Furthermore, one or more of the steps may be optional for particular implementations of the method.

Method 20 may include connecting transfection materials to carriers having codes, as shown at 22. The carriers may correspond to at least two classes, with each class having a different code or set of codes. Accordingly, a different transfection material may be connected to each class so that the different code or set of codes identifies each different transfection material. Such connections of transfection materials to coded carriers provide coded transfection materials. Each transfection material may include one or more nucleic acids. Each nucleic acid may be configured to decrease expression of a target gene, express a protein of interest, introduce a mutation into cells (such as into a preselected target gene), and/or provide an exogenous reporter gene activity, among others. Further aspects of carriers, codes, transfection materials, and connection of transfection materials are described below in Sections I-III.

Method 20 may include mixing the carriers, as shown at 24. The step of mixing may mix the two or more classes to position the carriers randomly and/or arbitrarily relative to one another. However, the codes identify each class of carrier and the transfection material connected to the class, allowing resolution of effects of the transfection materials on different subsets within a set of cells. The step of mixing may combine different coded transfection materials to create positional and/or nonpositional (positionally unconstrained) arrays (or mixtures) of transfection materials. Mixing may be optional in some embodiments of method 20. Further aspects of mixing carriers to form arrays, particularly library arrays of coded transfection materials, are described below in Section IV.

Method 20 may include placing the carriers in one or more compartments, as shown at 26. The carriers may be mixed before the step of placing, so that the step of placing may dispose portions of the array in each of a plurality of compartments. Alternatively, or in addition, the step of placing may form the array(s) within the compartment(s), for example, when different classes of carriers are combined in the compartment(s). Accordingly, the step of placing may be conducting before or after the step of connecting (22) and/or the step of mixing (24), among others. Exemplary compartments may include microplates (microtiter plates) with any suitable number of wells. Further aspects of placement of carriers are described below in Section V.

Cells may be apposed to the carriers, as shown at 28. The step of apposing may place the cells in proximity to the carriers, with individual subsets of the cells apposed to individual carriers. Apposing may add cells to the carriers, for example, in reverse transfection. Alternatively, or in addition, apposing may add the carriers to the cells, for example, in a forward transfection protocol in which the cells are connected first to a separate substrate before the carriers are added. In some embodiments, carriers may be added to a first set of cells that are substrate-connected, and then a second set of cells may be added to the carriers, to provide combined forward and reverse transfection. In any of these examples, apposed cells may become connected to the carriers or may be apposed but unconnected. The step of apposing may be conducted before, during, and/or after the steps of connecting, mixing, and/or placing. In some embodiments, the cells and a mixture of coded carriers may be provided, with the cells apposed to the mixture. These embodiments may have different carrier classes connected to different transfection materials. Further aspects of apposition of cells to carriers are described below in Section VI.

Method 20 may include introducing each different transfection material (or a subset thereof) into one or more cells (member cells) within a subset of the cells from a corresponding carrier apposed to the subset, as shown at 30. The step of introducing may transfect one or more apposed cells within each of the apposed subsets based on the proximity of carriers and their connected transfection materials to the subsets. The step of introducing may transfect one or more apposed member cells with each transfection material connected to one or more carriers of a class. Furthermore, the step of introducing may result in release of at least a portion of the transfection material connected to a carrier so that at least some of the released portion is introduced into one or more member cells of the apposed subset. Accordingly, the step of connecting (22) may provide a noncovalent connection between the carriers and the transfection materials. Further aspects of introduction of transfection materials into cells are described below in Section VII.

Method 20 may include exposing the cells to a modulator(s), as shown at 32. The modulator may be a candidate modulator being tested for a measurable effect, if any, on one or more transfected member cells and/or apposed subsets. Exemplary candidate modulators may include ligands, compounds, etc. In some embodiments, the carriers are placed in a plurality of compartments with different cell populations, and the cell populations are exposed to different modulators to screen the modulators. Addition of a modulator is an optional step of method 20. Further aspects of exposure to modulators are described below in Section VIII.

Method 20 may include measuring a characteristic of one or more apposed member cells and/or subsets of cells into which the transfection materials were introduced, as shown at 34. The characteristic may be any measurable property or behavior of each apposed member cell(s) and/or subset. In some embodiments, a characteristic of at least one apposed member cell and/or subset for each transfection material may be measured. When the cells have been exposed to a modulator, the characteristic measured may be related to presence of the modulator. Further aspects of measurement of cell characteristics are described below in Section IX.

Method 20 also may include reading the code of the corresponding carrier to identify the transfection material introduced into at least one apposed member cell and/or subset, as shown at 36. The identification of the transfection material may allow the measured characteristic of an apposed member cell(s) and/or subset to be related to the identified transfection material. Further aspects of reading codes are described below in Section IX.

Further aspects of the invention are described in the following sections: (I) coded carriers, (II) transfection materials, (III) connection of transfection materials to coded carriers, (IV) mixture of transfection materials to form arrays, (V) placement of carriers in compartments, (VI) apposition of cells to coded carriers, (VII) introduction of transfection materials into cells, (VIII) exposure of cells to modulators, (IX) measurement of cell characteristics and reading codes, and (X) examples.

I. Coded Carriers

The coded carriers generally comprise populations of particles, distinguishable at least in part by a detectable code. The carriers may support or hold transfection materials, reagents, and/or cells by connection of the carriers to the transfection materials, reagents, and/or cells, as described below. The carriers thus may serve to form a three-way proximity relationship between transfection materials, cells, and codes. Accordingly, each code may identify the connected transfection material and/or apposed member cell(s)/subset of cells, among others.

Carriers may have a composition that includes glass, plastic (for example, acrylates, such as poly(ethylmethacrylate) or PEMA), ceramic, sol-gel material, metal, protein, nucleic acid, lipid, and/or polysaccharide, among others. The material may be a solid, a gel or other porous material, or a combination thereof. In some embodiments, the carriers include a core portion, such as glass or plastic, among others, and a transfection material connected to the core portion. Accordingly, the core portion may include the code and may be inanimate.

The carriers or particles generally may have any suitable size. Preferred properties are determined by the application. For example, preferred sizes may be determined in part by what the carriers are connected to and identify, with carriers preferably being at least a few times larger than the molecules, organelles, viruses, cells, and/or so on that the carriers may be connected to and support. Preferred sizes also may be determined in part by the detection method, with carriers preferably being (at least for optical detection) larger than the wavelength of light but smaller than the field of view. Preferred sizes provide carriers termed microcarriers. Microcarriers may range between about ten microns and about four millimeters in diameter (or length). Alternatively, or in addition, microcarriers may have a length related to the cells that appose the carriers in an analysis, with the average length of the microcarriers being greater than an average diameter of the cells or between about one to fifty cell diameters.

Numerous applications of the invention may be carried out in microplates, such as 96, 384, or 1536 well microplates, or similar sample holders, having a relatively high density of relatively low volume wells. In these applications, the microcarriers preferably should be small enough so that at least two or more microcarriers may be viewed in the well simultaneously. Therefore, the maximum size dimension for microcarriers sometimes may be dictated by the well dimension in a specific microplate configuration or density. Conversely, the minimum area of microcarriers preferably should be large enough to support at least one cell. Thus, microcarriers for multiplexed cellular experiments may have an area of at least about 100 square microns.

Preferred carrier geometries may include at least substantially planar, for example, in the form of a wafer or sheet, and at least substantially cylindrical. The wafer or sheet may be square, rectangular, polygonal, circular, elliptical, and/or curvilinear, among others, when viewed from the top, side, or end, and may have at least one pair of opposing surfaces that are generally parallel. In some embodiments, at least one surface provides an experimental platform for connecting transfection materials and for apposition to cells.

Coded carriers may have surface relief. The surface relief may include any deviation from a substantially planar or convexly contoured surface to form a projecting or recessed region of the surface. Exemplary surface relief may include one or more grooves, ridges, holes, bumps, depressions, dimples, and/or the like. Recessed surface regions may provide advantages over planar and/or continuously convex surfaces. For example, the recessed regions may retain cells more effectively by, for example, providing a better gripping surface for the cells and/or minimizing fluid flow and/or turbulence near the surface region during carrier manipulations in fluid. In some embodiments, the recessed regions are configured to at least partially receive one or more cells. Alternatively, or in addition, the recessed regions may increase the carrier surface area and/or may provide more effective access of fluid (and reagents) when a surface of the carrier abuts a generally complementary supporting surface, such as a planar substrate during forward transfection. In some embodiments, projections are included on carriers. Such projections may increase surface area, facilitate carrier manipulation, and/or provide identifying regions of the carriers, among others. When used with cells, the formation of a more three-dimensional surface (ridges, etc.) may improve the probability of forming attachments to the cells, relative to a more two-dimensional surface.

One or more grooves (and/or ridges) on a carrier surface may facilitate exposure of cells to fluid and reagents during forward transfection. For example, in forward transfection, cells may be disposed between a surface of a carrier and a flat substrate surface on which the carrier is supported. Grooves may define open-ended compartments in cooperation with the substrate surface. Cells may be at least partially or completely contained in the compartments. Because the compartments are open-ended, fluid and reagents may access the compartment more efficiently to contact the cells. Accordingly, such grooves may allow cells to be analyzed on two opposing (generally upward- and downward-facing) surfaces of a carrier (see Example 4). Grooves also may facilitate the transfer and handling of the carrier by disrupting long-range juxtapositions of the planar surface of the substrate and that of the carrier (loss of surface tension binding).

The code generally comprises any mechanism capable of distinguishing different carriers. The code may relate to overall features of the carriers. These features may include carrier size, shape, and composition. Alternatively, or in addition, the code may relate to subfeatures of the carrier. These subfeatures may be positional and/or nonpositional, meaning that the code is based on the presence, identities, amounts, and/or properties of materials at different positions in the carrier and/or at potentially the same position in the carrier, respectively. These positions may be random and/or predefined.

Exemplary positional and nonpositional codes may be optically detectable. Such codes may be formed by using materials that differ in how they generate and/or interact with light (i.e., electromagnetic radiation, particularly visible light, ultraviolet light, and infrared light), such as their absorption, fluorescence, diffraction, reflection, color (hue, saturation, and/or value), intrinsic polarization, chemiluminescence, bioluminescence, and/or any other optically distinct property or characteristic. Positional codes may be formed by positioning different amounts and/or types of materials at different positions in or on a carrier, for example, at spots, lines, concentric circles, and/or the like. These positional codes may be read by determining the identities, amounts, and/or other properties of the code materials at each code position, for example, by measuring intensity as a function of position. Nonpositional codes may be formed, for example, by using at least two different materials, potentially at the same position, where the materials differ in how they interact with light. These nonpositional codes may be read by determining the presence and/or other properties of signals from the different materials, for example, by measuring intensity as a function of wavelength for an optical code. In each case, the amounts, positions, and/or values may be relative or absolute. Moreover, different types of codes may be combined to form yet other types of codes. In some embodiments, the codes may be read directly by interrogation with light, without reacting or processing the carriers.

Codes may define classes of carriers. Each carrier class is defined by a different code or set of codes. Accordingly, carriers in different classes may have different transfection materials connected to the carriers. The different transfection materials are identified by the different code or set of codes.

Further aspects of coded carriers, including carriers and codes that may be suitable, are described in the patents and patent applications listed above under Cross-References, which are incorporated herein by reference, particularly U.S. patent applications Ser. No. 09/694,077, filed Oct. 19, 2000; Serial No. 10/120,900, filed Apr. 10, 2002; and Ser. No. 10/273,605, filed Oct. 18, 2002.

II. Transfection Materials

Transfection materials generally comprise any naturally and/or synthetically produced materials capable of being connected to coded carriers and at least partially released from the carriers to be introduced into cells. Transfection materials introduced into cells are disposed at least partially in the interior of the cells, that is, at least partially inside the outer membrane of the cells. Exemplary regions within the interior include the nucleus, the cytoplasm, an organelle, an internal membrane, and/or the like. Transfection materials may produce, modify, eliminate, and/or report a cell characteristic, or may have no effect on the cell characteristic, among others.

Transfection materials may be nucleic acids or mixtures of nucleic acids, such as deoxyribonucleotide (DNA) polymers, ribonucleotide (RNA) polymers, nucleic acid analogs, and/or derivatives thereof. Exemplary nucleic acid analogs may include peptide nucleic acids (PNAs), that is, nucleotides joined by peptide bonds. The nucleic acids may have any suitable chain length; may be single-, double-, and/or triple-stranded; may be linear, branched, and/or circular; and/or may be connected covalently or noncovalently to any suitable compounds or moieties, such as proteins, lipids, dyes, enzymes, carbohydrates, etc.

Transfection materials may be one or more materials that are not nucleic acids. Examples of such other transfection materials may include proteins; peptides; chemical compounds or complexes; carbohydrates; viruses; prions such as CJD (Creutzfeld-Jacob disease), GSS (Gerstmann-Straussler-Scheinker syndrome), FFI (Fatal Familial Insomnia), kuru, and Alpers syndrome; and/or the like.

The transfection material may be, or may encode and direct expression of, an effector and/or a reporter, as described below. The effector or reporter gene may be synthetic and/or may be included in a vector capable of being propagated in bacteria or other suitable host. The vector may include additional genes and control sequences to provide a selectable marker(s)/drug resistance, replication origin(s), effector expression, and/or the like. An exemplary effector and/or reporter gene vector is a DNA plasmid with features that function in eukaryotic and/or prokaryotic cells.

Each transfection material may include two or more materials that are connected to each carrier and apposed to cells. For example, cells may be apposed to one or more nucleic acids that are or encode a known or candidate effector and one or more reporter genes. Because cells generally may be co-transfected with several transfection materials, any combination of transfection materials may be suitable.

A. Effectors

An effector generally comprises any material known to induce, and/or being tested for its ability to induce, a detectable response, such as a phenotypic change and/or a binding interaction, in cells. The effector may be a protein of interest, an RNA, and/or a DNA.

Effector proteins may be forward or reverse-transfected directly or expressed from transfection materials. Such effector proteins may be full-length or fragments of full-length proteins, either wild type or mutant. Exemplary effector proteins include receptors, ligands, enzymes, substrates, transporters, transcription factors, structural proteins, regulators, and/or the like.

Effector RNAs may be transfected directly or expressed from transfection materials, and may be antisense, sense, and/or at least partially double-stranded. Directly transfected RNAs may be produced by chemical or enzymatic synthesis in vitro, or by biosynthesis in vivo.

Effector RNAs may be antisense RNAs. Antisense RNAs generally include any RNA that is complementary to, or base pairs with, at least a portion of an RNA that is expressed in vivo, such as by a cell or a virus. Such antisense RNAs may be used to block expression of protein from, or another function of, sense RNAs expressed in cells. Exemplary antisense RNAs may include antisense transcripts encoded by DNA or RNA vectors, such as a plasmid or a virus. Other exemplary antisense RNAs may be synthesized or biosynthesized prior to transfection, as described above.

Effector RNAs may be sense RNAs. In some embodiments, sense RNAs may perform a function independent from base-pairing, such as encoding a protein or peptide; acting structurally, for example in a ribonucleoprotein complex; acting enzymatically; and/or the like. Such sense RNAs may correspond directly to portions of RNAs expressed naturally in vivo, or may be designed de novo. Exemplary sense RNAs include polypeptide-encoding RNAs that are translated to form full-length proteins, protein fragments, peptides, chimeras, mutants, deletions, and/or the like; structural RNAs, such as rRNAs, tRNAs, snRNAs, hnRNAs, and RNAs in other ribonucleoprotein or multi-component complexes; ribozymes; and/or the like.

Effector RNAs may be partially or fully double-stranded RNAs. Such partially or fully double-stranded RNAs may perform a structural role, may be translated, and/or may block expression through posttranscriptional gene silencing (PTGS). Effector RNAs that initiate and/or mediate PTGS, termed PTGS agents, typically are double-stranded RNA derivatives (including RNAi and small interfering RNA (siRNA), among others) that are synthetically or enzymatically derived, and/or encoded by transfected nucleic acid. Encoded double-stranded RNAs may be expressed as inverted repeats on transcripts that anneal intramolecularly, or as separate transcripts that anneal intermolecularly.

Effector DNAs may be forward and/or reverse transfected into cells using coded carriers. Effector DNAs may be synthesized chemically, enzymatically, and/or in vivo source, such as by cells or viruses. Exemplary effector DNAs include synthetic antisense oligonucleotides and oligonucleotide derivatives. Other exemplary effector DNAs may include structural or enzymatic DNAs. Furthermore, effector DNAs may include protein-binding sites for transcription factors or other cellular proteins, for example, to titrate such factors from endogenous binding sites.

B. Reporters

A reporter gene generally comprises any polynucleotide capable of reporting directly or indirectly on an aspect of an assay. The reporter gene typically includes (1) a regulatory region, and (2) a transcribed region encoding an expressed RNA and/or a protein reporter.

The regulatory region may include any control sequences that help to determine frequency or speed of reporter gene transcriptional initiation, elongation, and/or termination, and/or related aspects of translation. In turn, the control sequence may include a complex or simple enhancer, TATA box, initiator site, transcription factor binding element, RNA structural determinant, and/or RNA polymerase or cofactor interaction site, among others. Exemplary control sequences include promoters and promoter fragments from characterized genes and/or viruses (beta-actin, CMV, RSV, SV40, and so on). Other exemplary control sequences include synthetic binding sites for regulated or constitutively active transcription factors, such as nuclear hormone receptors (ecdysone-, dexamethasone-, or estrogen-responsive, among others), interferon regulated factors, metal response factors, SP1, CREB, AP-1, NF-kappaB, and the like. Control sequences may respond to the activity of a receptor class, such as G-protein coupled receptors (i.e., GPCRs, or “seven-pass transmembrane proteins”), interleukin receptors, and/or nuclear hormone receptors, among others.

The reporter material may be an RNA and/or a protein that reports or measures a characteristic of transfected cells. Reporter RNAs may be coding sequences, noncoding sequences, and/or arbitrary sequences. Alternatively, or in addition, the level or activity of reporter proteins may report a cell characteristic. A cell characteristic also may be revealed by changes in the physical location of a protein (or RNA) within or about a cell. Exemplary reporter proteins are readily detectable by intrinsic fluorescence, enzyme activity, and/or one or more other measurable properties. Examples of reporter proteins include green fluorescent protein (GFP), beta-galactosidase, chloramphenicol acetyltransferase, and luciferase.

Further aspects of transfection materials are described in the patents and patent applications listed above under Cross-References, which are incorporated herein by reference, particularly U.S. patent application Ser. No. 10/120,900, filed Apr. 10, 2002.

III. Connection of Transfection Materials to Coded Carriers

Transfection materials may be connected to coded carriers. This connection may allow the code on a carrier to identify the transfection material, as to its sequence, structure, and/or origin, among others.

Connection of a transfection material to a class of one or more carriers may be conducted by mixing or otherwise exposing the transfection material(s) to the carriers. Formation of the connection may be facilitated by any suitable treatment including, but not limited to, desiccation, heat, light, radiation, chemical reaction, and/or treatment with an adhesive (described below). Connection also may include treatment of the transfection material with a transfection vehicle, as described below, either before, during, and/or after connection of the transfection material to the carriers. After separate connection of different carrier classes to different transfection materials, the different classes may be placed together in a single compartment, such as a vial or a well of a microplate, to produce a mixture or positionally unconstrained array of transfection materials. Within the array, different classes of carriers connected to different transfection materials may include different adhesives (also termed adhesion promoters) and/or transfection vehicles. For example, a set of carriers may include a class of carriers connected to a peptide and another class connected to an RNA. In this case, each class may use different adhesives and/or different vehicles that are suited for transfection of either the peptide or the RNA.

Connection of transfection materials to carriers may require a balancing act between stable and unstable interactions. Generally, the connection should be sufficiently stable to retain a portion of the transfection material connected to the carrier during apposition of cells to the carrier in fluid. Accordingly, the connection may be covalent to stabilize the connection. However, in some embodiments the connection may be transient enough that the transfection material is at least partially released for introduction into one or more cells apposed to the carrier. Accordingly, the connection may be noncovalent. In any case, the transfection materials may be configured to be released at least partially when exposed to fluid (such as fluid of a physiological ionic composition and/or pH) and/or a competing binder, and/or when apposed to cells.

The stability of a noncovalent connection may be facilitated by one or more adhesives included in a coded carrier. As used herein, an adhesive is any material that improves connection of transfection materials to coded carriers. Adhesives may be connected covalently or noncovalently to the carriers, before, during, and/or after connection of the carrier to the transfection material. Examples of adhesives may include am inosilane; polylysine; gelatin; atelocollagen; polyethylenimine; a dendrimer, such as a cationic or amphipathic dendrimer (for example, an activated-dendrimer available from QIAGEN); an extracellular matrix component, mixture, or extract; serum albumin; a nucleic acid binding protein or other macromolecule, including sequence-specific or—nonspecific DNA and/or RNA binding proteins; compounds that bind nucleic acids, such as intercalating agents (ethidium monoazide, ethidium bromide, etc.) or major or minor groove-binder agents that bind to the major or minor groove of a nucleic acid duplex, respectively; nucleic acids, such as single- or double-stranded DNA or RNA that form hydrogen bonds with the transfection material; and/or the like. Alternatively, or in addition, carriers may be rendered more adhesive by etching and/or plasma treatments (that is, electronic discharge with or without gas present). Adhesives may be connected directly to the carrier, or connected indirectly through a bridge. In some embodiments, the bridge may be formed by materials (compounds, polymers, biomolecules, etc.) that react with, or bind to, both the carrier and the transfection material, such as aminosilane, polylysine, and/or the like.

In some cases, the carrier and the transfection material may be connected covalently or in a relatively stable noncovalent connection. Such covalent connection or relatively stable noncovalent connection may be suitable, for example, if a treatment of the carriers, or a releasing activity produced by the cells, is capable of breaking or destabilizing the covalent or stable noncovalent connection. Such carrier treatments or releasing activities produced by cells may target the transfection material directly, the carrier, an adhesive that holds the transfection material, and/or any chemical bond(s) or forces that join them. Carrier treatments may include exposure to chemicals (such as changes in pH or ionic strength, catalysts, enzymes, metals, chelates, etc.) and/or exposure to physical conditions (such as changes in temperature, exposure to light or emitted particles, etc.). Accordingly, transfection materials may be connected to carriers using specific binding pairs, such as those listed below in Table 1 of Section VI. The transfection material may be connected covalently or noncovalently to a specific binding member.

The ability of cells to produce a releasing activity that at least partially releases the transfection material may be a natural or engineered property of the cells. Naturally expressed releasing activities may correspond to enzymes normally secreted by cells, such as enzymes that cleave bonds present in proteins, carbohydrates, proteoglycans, nucleic acids, lipids, extracellular matrix components, etc. Such releasing activities that are naturally expressed may be secreted by each cell within an apposed subset of cells or by less than all of the subset, to promote nonselective or selective transfection, respectively. In some embodiments, cells may release transfection materials from virus particles connected to the carrier by infection. Alternatively, transfection materials may be released by releasing activities expressed in (and secreted by) cells as a result of experimental manipulation. For example, the cells may be engineered to express a secreted cleavage enzyme that releases the transfection material from the carrier. Such a secreted enzyme may be a modified derivative that includes a secretion signal to promote exit from the cells.

In some embodiments, the releasing activity, whether expressed naturally or by experimental manipulation, may cleave an adhesive that is connected to the transfection material either covalently or noncovalently. For example, nucleic acid transfection materials may be tethered to galacturonic acid, which is fused to an adhesive or which acts as an adhesive directly. In this case, cells that express extracellular galacturonidase may cleave galacturonic acid and locally release tethered nucleic acid for carrier-localized transfection. Thus, particular cells within a carrier-apposed subset may be selectively transfected based on expression by the particular cells of a releasing activity directed against the transfection material, an adhesive, or a bridging group that links either to the carrier.

Further aspects of connecting (or “associating”) transfection materials to (or with) coded carriers are described in more detail in the patents and patent applications identified above under Cross-References, which are incorporated herein by reference, particularly U.S. patent application Ser. No. 10/120,900, filed Apr. 10, 2002.

IV. Mixture of Transfection Materials to Form Arrays

Two or more classes of carriers and a different transfection material connected to each class may be mixed to produce an array or mixture of transfection materials. The different transfection materials in the array are structurally distinct, for example, having different nucleotide sequences with different nucleic acids or different amino acid sequences with different proteins, among others. The different transfection materials may provide a library.

The library has two or more members and usually includes a property that is related between the members. For example, a library may encode, or include, a family of structurally and/or functionally related proteins, such as different nuclear hormone receptors, G-protein coupled receptors (GPCRs), ion channels, cytoskeletal components, cell-cycle regulators, etc. Alternatively, or in addition, the library may encode wild-type and/or mutant derivatives of a protein. In other embodiments, the library may include different defined or random complementary or genomic DNAs from an organism, tissue, cell type, patient sample, disease state, developmental stage, tumor, blood, genetic background, and the like, and each complementary or genomic DNA may have known or unknown structure and/or function. Similarly, the library may contain RNAs corresponding to these complementary or genomic DNAs or fragments thereof. Alternatively, or in addition, the library may include antisense RNAs or PTGS agents (dsRNA, RNAi, etc.) directed against target genes/RNAs of interest. In yet other embodiments, the library may encode two or more reporter genes with distinct regulatory sequences and/or reporter sequences. In still further embodiments, the library may include a set of antisense nucleic acids or expression vectors, or PTGS agents, directed against transcripts of a common gene, a set of related genes, and/or a set of diverse, unrelated genes.

In some cases, members of a library may be relatively or completely unrelated. For example, a library may include one or more different nucleic acids and one or more different peptides, one or more different DNA expression vectors and one or more different RNA molecules, one or more different DNA duplexes and one or more different single-stranded DNA molecules, and/or so on.

Coded arrays/libraries may be formed from coded carriers and their connected transfection materials. The arrays may be nonpositional, positional, or partially positional. In a nonpositional array, each transfection material may be identified by a connected code only and independent of carrier position. Thus, nonpositional arrays may be formed by arbitrarily and/or randomly positioning different transfection materials and their carriers relative to each other, that is, by forming a mixture of the carriers. In the mixture, members of different classes of carriers may have positions that are fixed or variable. In a positional array, each transfection material may be identified based on the absolute and/or relative position of the carrier to which the transfection material is connected. An exemplary positional array is a kit including two or more classes of carriers with a different transfection material connected to each class. In a partially positional array, each transfection material may be identified based on a combination of the code and the position of the carrier. Thus, partially positional arrays may use one code to identify two or more different transfection materials positioned at different positions, such as in different wells of a microplate. Further aspects of nonpositional, positional, and partially positional arrays are described in the patents and patent applications listed above under Cross-References, which are incorporated herein by reference, particularly U.S. patent application Ser. No. 10/120,900, filed Apr. 10, 2002.

V. Placement of Carriers in Compartments

Placement of carriers generally includes any manipulation that disposes carriers in one or more compartments for further manipulation or assay. Different classes of carriers may be placed in a compartment as a mixture or individually. The carriers may be placed in the compartment before and/or after connection to a transfection material and/or apposition to cells. Accordingly, carriers may be placed into a compartment that already includes cells, reagents, transfection materials, fluid, or that is empty, among others. In some embodiments, the carriers may be placed in apposition to cells, with the carriers and cells substantially covered by fluid, such as an aqueous media.

Any suitable method may be used to place carriers in the compartments. The carriers may be placed while included in fluid, for example, with a pipet, or may be placed without fluid, such as with a spatula, scoop, or forceps. In some embodiments, the carriers may be placed by magnetic transfer. The carriers may be placed automatically or manually.

The carriers may be placed into any suitable compartment for transfection. The compartment may have walls, such as a well or a test tube, or may be a region of a surface, such as a portion of a microscope slide surface. In exemplary embodiments, the compartments are provided by wells of a microplate. The microplate may have any suitable number of wells, such as 96, 384, 1536, etc.

VI. Apposition, of Cells to Coded Carriers

Cells are apposed to coded carriers so that subsets of the cells are in apposition to individual coded carriers for transfection of one or more cells within each apposed subset. Apposition generally includes any suitable proximity relationship between cells and carriers, for example, preferably a proximity relationship capable of allowing and/or effecting transfection of at least one of the apposed cells. In some embodiments, the cells are present in numerical excess over the carriers, that is, the number of test cells is greater than the number of carriers.

Apposition places one or more subsets of the cells in apposition to a corresponding one or more individual carriers. The particular cell subset apposed to an individual carrier may be defined as the subset of the cells that is most closely apposed to the individual carrier relative to other carriers of a carrier mixture. Alternatively, the particular apposed subset may be defined as the cell subset that contacts the individual carrier, the cell subset separated from the individual carrier by less than a selected distance, and/or the cell subset that is connected to the individual carrier. The selected distance may be less than about one, two, or five cell diameters, among others. Alternatively, the particular cell subset may be defined by cells that overlap or are included within a footprint of the individual carrier. Other aspects of apposition between a subset of cells and a carrier, particularly for forward transfection, are described below in Example 3.

The apposition of cells and carriers may be based on any mechanism that holds the cells in proximity to the carriers until a characteristic(s) of the cells is measured (see below) and/or a code(s) is read. Accordingly, the apposition may be a stable or semi-stable connection of the cells to the carriers, so that the carriers carry some or all of the cells with them when they are moved. Alternatively, or in addition, apposition may be based on a static relationship between the carriers and a separate substrate to which the cells are connected. For example, in forward transfection the carriers may be held in place on the substrate by gravitational, frictional, magnetic, and/or electrical forces, among others. The substrate may be a contiguous surface, such as that provided by the interior of a microplate well or tissue culture dish, among others. The substrate may have any suitable shape, such as substantially planar or nonplanar (such as curved or semi-spherical, among others). Furthermore, the substrate may have a length or maximum linear dimension that is larger than the average length or maximum linear dimension of the carriers, preferably at least about two-fold or five-fold greater.

A cell population or subset of the cells may be connected to each carrier through direct and/or indirect molecular interactions. Accordingly, connection may be mediated by any suitable mechanism, including electrostatic interactions, covalent bonding, ionic bonding, hydrogen bonding, van der Waals interactions, electromotive force, fluidics, and/or hydrophobic/hydrophilic interactions, among others. In general, binding may be facilitated by the appropriate selection, treatment, and/or modification of the carriers and/or cells.

Connection of a cell population to an individual carrier may be facilitated by appropriate selection of the carrier material, geometry, and connection region. The cell population may be connected to external and/or internal regions of the carrier. Thus, the carrier may include a relatively flat or gently contoured external binding surface or may have a surface contour that facilitates binding and retention, such as recesses, grooves, a rough texture, etc. Moreover, the carrier may include a modifiable binding surface, so that the surface may be treated as desired to promote binding of the cell population to the carrier. Alternatively, the carrier may be a porous material, such as a gel or semi-solid matrix, which may allow at least a portion of the cell population to migrate inside or to be disposed otherwise in the interior of the carrier.

Connection of cells to carriers may be facilitated by appropriate treatment of the carriers, either before or after apposition to cells. Suitable treatments may include chemical reaction, charge modification, temperature changes, light, radiation, and/or desiccation, among others. Thus, in some applications, carrier surfaces may be pretreated or otherwise modified so that electrostatic or, in given cases, van der Waals or covalent binding of cells to carriers is promoted. For example, binding surfaces may be connected to a substrate material. A substrate material is any material that facilitates or improves connection of a cell to a carrier. Exemplary substrate materials may include poly-L-lysine, poly-D-lysine, or polyethylenimine.

Other exemplary substrate materials may be extracellular matrix material(s). The extracellular matrix material may be any component, mixture, portion, or all of any external matrix produced by cells and deposited external to the cells. An extracellular matrix component may be an extracellular matrix protein(s), a glycosaminoglcyan(s), and/or a mixture thereof. Examples of extracellular matrix proteins may include gelatin, collagen, laminin, fibronectin, entactin, vitronectin, fibrillin, elastin, and/or the like. Examples of glycosaminoglycans may include heparan sulfate, chondroitin sulfate, heparin, keratan sulfate, and/or hyaluronic acid, among others. The extracellular matrix material may be a complex mixture of compounds, such as an extracellular matrix produced by a cell(s), tissue(s), embryo(s), fetus(es), and/or organism(s), among others. In some embodiments, the substrate material may include an extracellular matrix component (or components) produced by cells apposed to, and then separated from, one or more carriers.

In some cases, connection of cells to carriers may be facilitated by interactions between specific binding pairs (SBPs), where one member of the pair is connected to cells and the other member of the pair is connected to carriers. The interactions between members of a specific binding pair typically are noncovalent, and the interactions may be readily reversible or essentially irreversible. An exemplary list of suitable specific binding pairs is shown in Table 1.

TABLE 1
Representative Specific Binding Pairs

| First SBP Member | Second SBP Member |
|---|---|
| antigen | antibody |
| biotin | avidin or streptavidin |
| carbohydrate | lectin or carbohydrate receptor |
| DNA | antisense DNA |
| enzyme substrate | enzyme |
| histidine | NTA (nitrilotriacetic acid) |
| IgG | protein A or protein G |
| RNA | antisense RNA |
| receptor or chemical chelate | ligand |

Connection of cells to carriers also may be facilitated by appropriate selection and/or treatment of the medium in which the cells and carriers are apposed. For example, the medium may include binding mediators that participate in or otherwise promote interactions between cells and carriers, for example, by forming cross-bridges between the cells and carriers and/or by counteracting the effects of binding inhibitors associated with the cells and carriers. The binding mediators may act specifically, for example, by binding to specific groups or molecules on the cells and carriers. Thus, biotin might act as a specific binding mediator by binding to and cross-linking avidin or streptavidin on the cells and carriers. The binding mediators also may act less specifically, or nonspecifically, for example, by binding to classes or categories of groups or molecules on the cells and carriers. Thus, calcium ions might act as a relatively nonspecific binding mediator by binding to and cross-linking negative charges on the cells and carriers.

Cells may be connected indirectly to a carrier. For example, indirect connection of cells to carriers may be mediated by a reagent, for example, by binding of the cells to a ligand, a cell-selector, or other material that is or will be connected to the carriers. Connection may facilitate subsequent analysis of the cells. Alternatively, the presence, absence, or level of connection to, or binding of, cells to carriers through a reagent may provide a cell characteristic.

Cells may be placed in apposition to carriers by any suitable method. In some embodiments, cells may be mixed with carriers, allowing the cells to connect to any available surfaces of the carriers. In other embodiments, connection may be at least substantially restricted to one or several surfaces of the carriers. The cells may be apposed to the carriers so that the cells selectively encounter and thus connect to a portion of the carrier. For example, carriers may be distributed randomly, but substantially in a monolayer, on a horizontal surface, such as the bottom of a tissue culture container or microplate well. Cells in suspension may be added to the container or well and allowed to settle onto an upwardly facing surface of the carriers, where they may connect. Alternatively, or in addition, cells may be connected first to a separate substrate, and then the carriers and their connected transfection materials placed adjacent the substrate and thus adjacent the substrate-connected cells.

Connection of cells to carriers may be conducted with the carriers provided in a positional array, for example, by arranging or forming the carriers on a substrate. Individual cell populations may be connected to carriers within the array. After connection of cells to the array, carrier distribution may be randomized to arbitrarily position the carriers. This approach may allow a more economical use of limited numbers of cells, for example, from a patient sample.

Further aspects of apposing and/or connecting (or “associating”) cells to (or with) carriers, including different types of cells that may be suitable, are described in the patents and patent applications identified above under Cross-References, which are incorporated herein by reference, particularly U.S. patent application Ser. No. 10/120,900, filed Apr. 10, 2002.

VII. Introduction of Transfection Materials into Cells

Transfection of cells is localized by carriers that are connected to and support transfection materials. One or more member cells apposed to each carrier is transfected selectively with the transfection material connected to the carrier, generally by release of the transfection material.

Transfection of adherent or substrate-attachable cells may be conducted with coded carriers by forward transfection, reverse transfection, or both in a multiplexed experiment. Forward and reverse transfection of cells are distinguished here by whether the cells are attached to a separate substrate or not when placed together with coded carriers for transfection. In forward transfection, cells first are connected to a substrate and then are placed together with coded carriers. By contrast, in reverse transfection, cells are not attached to a substrate when first placed together with coded carriers. In either type of transfection, the transfection material connected to each carrier is selectively directed, based on proximity, to one or more apposed cells provided by an apposed cell subset. The transfection material is introduced into the one or more cells, transfecting the one or more cells. Based on the efficiency of transfection, any number of cells with an apposed subset may have transfection material introduced, up to all cells of the subset. Carrier-localized forward and reverse transfections are described further in the Examples of Section X below.

Transfection may be stable or transient. Stable transfection results in stable maintenance of the transfected materials in cells during division, through genome integration or episomal replication, among others. Transient transfection generally introduces transfected material temporarily. Transiently introduced material may be diluted and/or degraded with continued cell incubation.

Transfection materials may be introduced into cells by transfection using any transport/transfection vehicles or treatments capable of promoting or facilitating internalization of transfection materials. Exemplary transfection vehicles include a particulate carrier, such as a calcium phosphate co-precipitate; a vesicle or micelle, such as a liposome; a virus or phage (or portions thereof, such as coat proteins); polyethylene glycol; a peptide carrier; atelocollagen; polyethylenimine; projectiles, such as fired by a gene gun; a dendrimer such as the SUPERFECT transfection reagent available from QIAGEN; and/or the like. Exemplary transfection treatments include injection, carrier bombardment, heat, shock loading, and/or electoporation. Suitable vehicles or treatments may be selected according to the properties of the transfection materials and/or cells being transfected, among others, and may be different for some of the transfection materials in an array.

Further aspects of transfection are described in the patents and patent applications identified above under Cross-References, which are incorporated herein by reference, particularly U.S. patent application Ser. No. 10/120,900, filed Apr. 10, 2002.

VIII. Exposure of Cells to Modulators

Cells (and/or carriers/transfection materials) may be exposed to modulators, before, during, and/or after transfection. Modulators, also termed candidate modulators when the modulators arc being tested for their effects, generally comprise any chemical, biological, and/or physical agent or treatment that has the potential to affect cells, carriers, and/or transfection materials, as described below.

Modulators may be chemical modulators, including any synthetic or naturally occurring element, molecule, polymer, complex, covalently linked molecules or polymers, noncovalently linked molecules or polymers, or heterogeneous multi-constituent assembly, or mixture thereof. Examples of chemical modulators may include compounds with known or suspected biological activity; ligands; antibodies; single-, double- or triple-stranded, linear, branched, or circular, naturally occurring or synthetic DNA or RNA molecules; synthetic anti-sense oligonucleotides, including derivatives or analogs, such as peptide nucleic acids; double stranded and/or interfering RNAs; peptides or peptide libraries; lipids; carbohydrates; and/or proteins or protein mixtures. Chemical modulators may include enzymes, especially proteases like stromelysin, tissue plasminogen activator (tPA), urokinase plasminogen activator (uPA), plasmin, elastase, gelatinase, matrilysin, collagenase, and so on, which act on the extracellular matrix, or inhibitors of these proteins (such as tissue inhibitors of metalloproteases, or TIMPs). Other exemplary chemical modulators may include hormones that interact with, or are included in, extracellular matrices, such as fibroblast growth factors, among others. Chemical modulators also may include general media composition, such as ambient gas composition, ionic strength, pH, ionic composition, divalent cation concentration (e.g., Ca²⁺ concentration), and/or nutrient mixture.

Modulators may be biological modulators. Biological modulators include biological entities, or fragments or extracts thereof. Examples of biological modulators include prokaryotic or eukaryotic cells, viruses, cell fragments, or extracts from cells, tissues, organisms, or embryos. Other biological modulators may include an expression library. Expression libraries generally comprise any library formed from cells, where members of the library express a foreign material or overexpress an endogenous material. Examples of expression libraries include phage libraries, such as phage display libraries that exhibit antibodies, receptors, or ligands; bacterial libraries in which foreign nucleic acid sequences are expressed; and eukaryotic cell libraries formed from cDNA or genomic expression libraries or other expression vectors.

Modulators may be physical modulators. Physical modulators include any environmental condition or treatment. Examples of physical modulators include heat; pressure; a gravitational field; an electric and/or magnetic field; light (including UV, visible, infrared, and other forms of electromagnetic radiation); shear; motion; contact with another object (such as another cell, a substrate, implanted medical device, etc.); and/or the like.

Cells, transfection materials, and/or carriers may be exposed to one or more modulators, before, during, and/or after apposing the cells to the carriers. The cells transfection materials, and/or carriers generally may be exposed to modulators for any suitable duration and at any suitable time point or interval during an analysis. Modulators may be candidate modulators that are being tested for their activity on transfected cells. Exemplary candidate modulators include drug candidates used to carry out a drug screen. Carrier-localized transfection may provide a direct or indirect target for a drug screen. Thus, any effect on the target may be a measurable characteristic of the transfected cell population apposed to a coded carrier. For example, the target may be a reporter gene whose activity may be regulated by the modulator. Alternatively, or in addition, the target may be directly bound by the modulator. For example, the target may be a receptor whose activity may be inhibited or activated by the modulator.

Exemplary modulators are described in more detail in the patents and patent applications identified above under Cross-References, which are incorporated herein by reference, particularly U.S. patent application Ser. No. 10/120,900, filed Apr. 10, 2002.

IX. Measurement of Cell Characteristics and Reading Codes

Cell characteristics may be measured and codes may be read at any time during a multiplexed cell transfection with coded carriers. Measurement of cell characteristics and reading codes may be performed in any order and on any number of transfected subsets of cells and carriers. Moreover, these steps generally may be performed using any suitable examination site, such as a slide, a microplate, or a capillary tube, and any suitable detection device, such as a microscope, a CCD array, an optical sensor, a film scanner, or a plate reader.

The cell characteristic may be measured from the subset of cells that is apposed to a carrier. Accordingly, the characteristic may relate to individual cells of the subset, subcellular regions of the subset, or extracellular regions adjacent individual cells of the subset. The characteristic may be measured from only one cell or region apposed to a carrier, from less than all cells or regions apposed to a carrier, and/or from all cells or regions apposed to a carrier. The efficiency of transfection may determine the percentage of the subset into which the transfection material is introduced. Accordingly, the cell characteristic may be measured selectively from detectably transfected members of the subset or nonselectively from some or all cells of the subset, regardless of whether they are detectably transfected or not. As a result, the characteristic may be measured as a combined value or average from both transfected and untransfected cells apposed to a carrier.

The characteristic may be any molecular, cellular, and/or extracellular aspect measured from the subset of cells apposed to a carrier. The molecular characteristic may relate to a cellular constituent, such as the number, concentration, distribution, presence/absence, partnership, structure, modification, or activity (such as enzyme activity or binding activity) of the constituent. Exemplary constituents may include a nucleic acid, protein, ion (for example, to measure calcium flux), lipid, carbohydrate, metabolite, etc. Exemplary proteins may include reporter proteins that are encoded by the transfection material (such as beta-galactosidase, GFP, luciferase, chloramphenicol acetyl transferase, etc.). The constituents may be endogenous, transfected, or encoded, among others. Cellular aspects may include any measurable cellular or subcellular phenotype, such as cell proliferation, reporter gene activity, cell cycle distribution, DNA synthesis, nuclear import, signal transduction, differentiation, transcription, morphology, apoptosis, import, export, subcellular transport, electrical activity, and/or the like.

Cell characteristics may be measured adjacent any suitable surface or surfaces of the carrier to which the subset is apposed. The subset may be apposed to one surface of the carrier, opposing surfaces of the carrier, and/or to any selected subset or set of surfaces of the carrier, among others. Accordingly, cell characteristics may be measured for a subset of cells apposed to one carrier surface, opposing surfaces, and/or near any region of the carrier In some embodiments, a cell characteristic(s) may be measured concurrently from cells near each of two opposing surfaces, for example, by choosing a suitable microscope objective. The cells may be forward-transfected near one surface and reverse-transfected near another surface, as described further in Example 4. Furthermore, different types of cells or different transfection materials may be apposed to distinct surfaces of a carrier, such as opposing surfaces. Accordingly, the same or different types of characteristic(s) may be measured from cells near distinct carrier surfaces.

The code may be read before, during, and/or after measuring the cell characteristic. Reading the code may include discerning or determining a positional and/or nonpositional code of a carrier by any suitable approach, such as optical and/or nonoptical techniques. Exemplary optical techniques include sensing light (particularly visible light, UV light, and infrared light) positionally or nonpositionally from a carrier. Exemplary nonoptical techniques may include electrical analysis of a carrier to read a nonoptical code, such as measurement of the carrier's capacitance, impedance, conductance, etc., in a positional or nonpositional fashion within the carrier. Whenever the code is read, it should be linked or linkable to the measured cell characteristic or interaction. This linkage may identify the transfection material associated with the carrier, and/or may identify other aspects related to the carrier, including the type of cells, the modulator exposed to the cells, other experimental parameters (such as reaction times or conditions), order of manipulations, and/or so on.

Additional exemplary cell characteristics, and methods for reading codes and measuring cell characteristics, are described in more detail in the patents and patent applications identified above under Cross-References, which are incorporated herein by this reference, particularly the following U.S. patent applications: Serial U.S. Pat. No. 09/694,077, filed Oct. 19, 2000; Ser. No. 10/120,900, filed Apr. 10, 2002; and Ser. No. 10/282,904, filed Oct. 28, 2002.

X. Examples

The following examples describe selected aspects and embodiments of the invention, including methods for forming arrays of transfection materials, forward/reverse transfection with such arrays, and exemplary experiments or screens that may be performed with these arrays. These examples are included for illustration and are not intended to limit or define the entire scope of the invention.

### EXAMPLE 1

Multiplexed Reverse Transfection of Cells

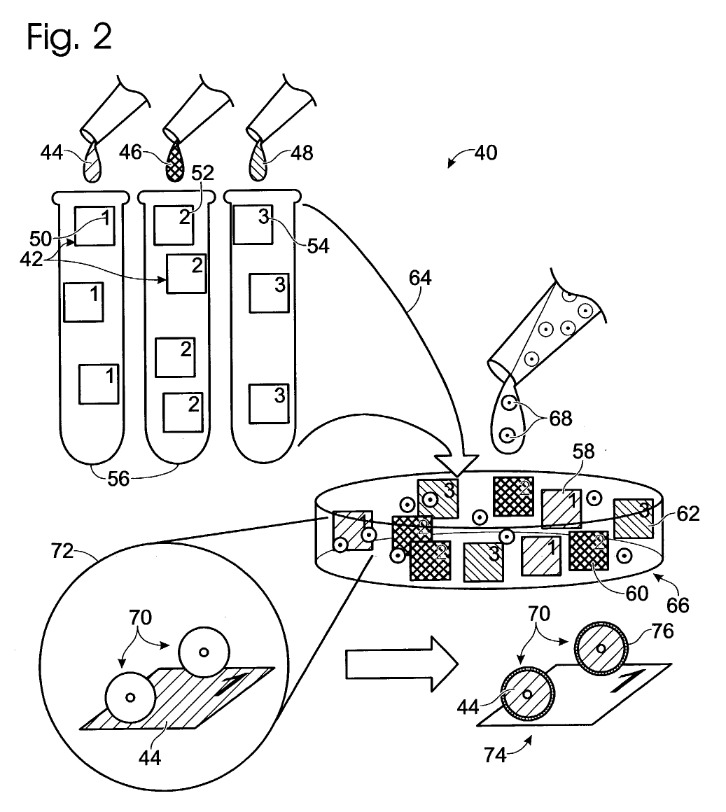

This example describes a method 40 for making and using a coded array of transfection materials. Here, the transfection materials are connected separately to carriers of different classes, mixed to arbitrarily distribute the transfection materials and their connected carriers, and then used to reverse transfect cells; see FIG. 2.

In method 40, different classes of coded carriers 42 are connected to different transfection materials 44, 46, 48. Here, the transfection materials are different DNA expression vectors encoding different receptor proteins. Each class of carrier may have a different code 50, 52, 54 (or codes) that identifies the connected transfection material. The carrier classes may be separated from one other in different compartments during connection of the different transfection materials to the carriers. For example, the different carrier classes may be placed in different locations on a substrate or in distinct containers 56, such as different test tubes, tissue culture plates or flasks, wells of a microplate, etc. Connection to the transfection materials may be conducted, for example, by coating the carriers with the transfection materials, either with the carriers arranged as a monolayer or piled randomly. Such connection may be described as producing coded transfection materials 58, 60, 62.

After connection, carriers 42 and their coded transfection materials may be mixed, shown at 64, to produce an array or mixture 66 of transfection materials connected to coded carriers. The identity of each transfection material in the array is maintained by code 50, 52, or 54 on each coded carrier to which the transfection material is connected. The transfection materials may be exposed to a transfection vehicle or treatment at any time during before, during, and/or after connection to the coded carriers.

Cells 68 may be apposed to coded carriers before, during, and/or after the carriers are connected to the transfection materials, and before and/or after the carriers are mixed to produce the array. Here, cells 68 are apposed to the carriers and their connected transfection materials after transfection materials have been mixed. The cells may be added, for example, as a suspension in fluid to allow the cells to settle on, and/or connect to, the carriers. Alternatively, or in addition, array 66 may be added to the cells, for example, with the cells connected first to a substrate separate from the array, as described below in Example 3. A single set of cells 68 may contribute cell populations that are connected to each coded carrier. Alternatively, other manipulations may be carried out, such as splitting or dividing array 66 into sibling arrays and connecting each sibling array to different cells or different types of cells.

The bottom of FIG. 2 shows magnified views of a subset 70 of cells before, shown at 72, and after, shown at 74, transfection with transfection material 44. Such transfection involves introduction of at least a portion of transfection material 44 into cell subset 70. The introduction may occur spontaneously and/or through the action of a transfection vehicle or treatment, as described above in Section VII. When subset 70 is connected to the carrier, as shown here, connection may be through interactions or bonds that are distinct from interactions with the transfection material. Transfection material 44 may enter subset 70 and may have an effect in the subset 70, based on the structure of the transfection material. Here, transfection material 44 directs expression of a cell surface protein 76, such as an integral membrane receptor (for example, a G-protein coupled receptor).

### EXAMPLE 2

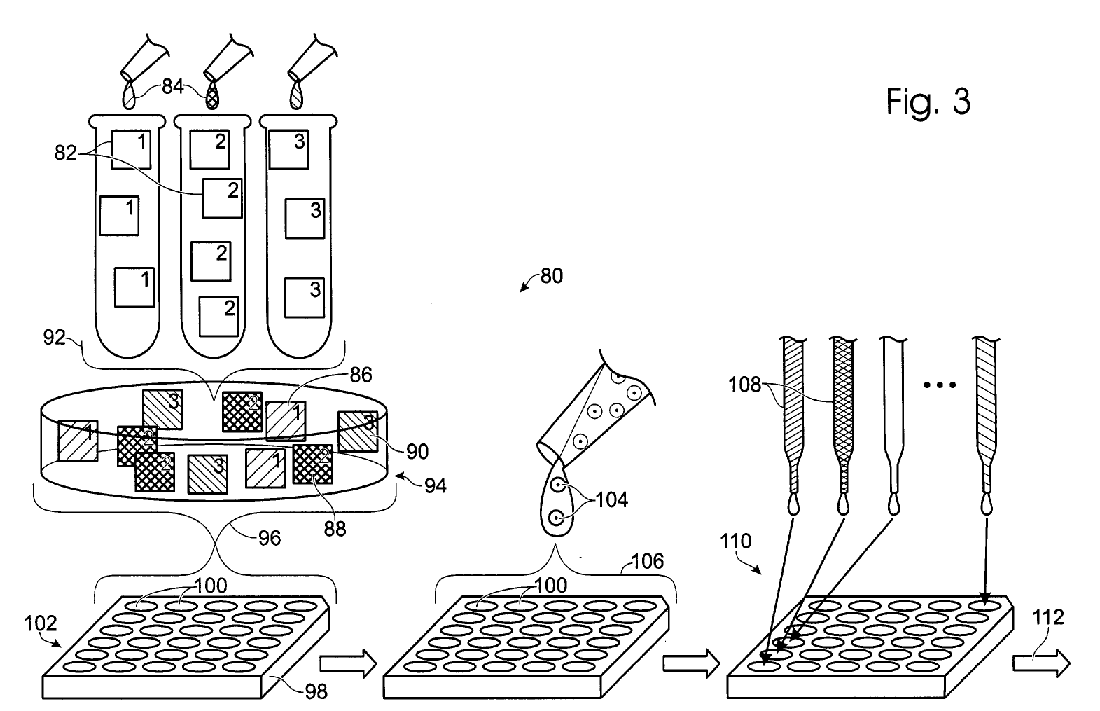

Screening Modulators using Reverse-Transfected Cell Arrays This example describes a method 80 using reverse-transfected cell arrays to screen modulators; see FIG. 3. Method 80 uses coded carriers to conduct multiplexed transfection of cells to test the action of the modulators on the cells.

Coded carriers 82 are connected to different transfection materials 84. The resulting coded transfection materials 86, 88, 90 may be combined, shown at 92, to produce a positionally unconstrained parent array 94 of coded transfection materials. Parent array 94 may be divided or split, shown at 96, by transferring portions of the array to a plurality of compartments, such as wells of a microplate 98. Each portion is a sibling array 100, each of which may have a similar representation of coded transfection materials as parent array 94. Together, sibling arrays 100 may provide a partially positional set 102. Alternatively, array 94 may be divided by transferring portions of parent array 94 into individually identifiable compartments, such as test tubes, that are not positionally constrained, but which are otherwise identifiable.

Partially positional set 102 may be apposed to cells 104 by combining a population of cells with each sibling array 100, shown at 106. The same and/or different types of cells may be used in each well, depending on whether the assay is intended to look at variations between types of cells or variations between modulators (or both). Here, portions of one population of cells are distributed to the wells of microplate 98, to be apposed to the distributed sibling arrays of coded transfection materials. In some embodiments, apposition of cells to carriers, or at least combining cells with carriers, may be carried out before transferring portions of parent array 94 to microplate 98 (shown at 96), or before combining individual coded transfection materials (shown at 92). Furthermore, in some embodiments, apposition of cells and coded carriers may be carried out prior to connecting transfection materials to the carriers. In some embodiments, the cells may be connected to wells of microplate 98 before the carriers are placed in the wells, to achieve forward transfection (see Example 3).

During and/or after apposing the cells to the carriers, transfection materials connected to the carriers may be introduced into subsets of the cells that are in apposition with individual carriers. Each different transfection material or less than all different transfection materials may be introduced into one or more subsets within a well, based on the efficiency of introduction, the distribution of cells within the well, the representation of transfection materials from the parent array within the well, and/or so on.

Before, during, and/or after transfection, cells may be exposed to candidate modulators 108, shown at 110. The identity of each modulator 108 may be tracked positionally during exposure, for example, based on the position of a well within microplate 98, or nonpositionally in an identifiable vessel. Alternatively, or in addition, the array may be marked internally, for example, with a distinct array- or modulator-identifying carrier. However, in some embodiments, the identity of each modulator may be carried by the code, particularly during subsequent manipulations that destroy positional array-identifying information.

After appropriate incubation with modulators, a characteristic of the transfected cell subsets may be measured, and the code may be read, as indicated at 112. In some embodiments, both a characteristic and a code may be measured and read, respectively, from each carrier analyzed. Alternatively, characteristics and/or codes may be measured and read selectively. For example, a carrier code may be read only when the characteristic measured for an apposed cell population is of interest, such as a desired response to a candidate modulator. Here, each well of the microplate may provide information about a modulator's effect on a plurality of different types of cell subsets that are produced by transfection with a corresponding plurality of different transfection materials.

### EXAMPLE 3

Multiplexed Forward Transfection using Coded Carriers

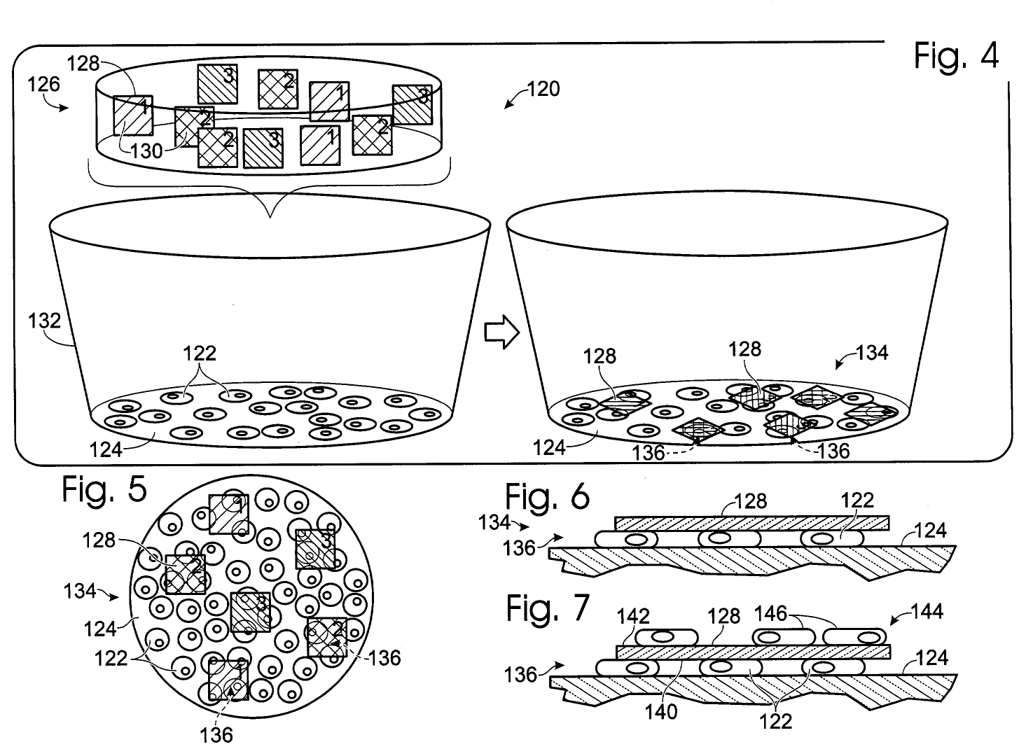

This example describes methods for forward transfection using coded carriers; see FIGS. 4-6.

FIG. 4 shows a method 120 for forward transfection using coded carriers. Method 120 may be carried out by connecting cells 122 to a substrate 124 before the cells are apposed to an array 126 of coded carriers 128 and their connected transfection materials 130. Here, cells 122 have been connected to substrate 124 provided by a well 132 of a microplate.

After the cells have adhered to a surface of the well, array 126 may be placed in the well, to appose the carriers to the cells in apposed position 134. The carriers (and their transfection materials) may be positioned arbitrarily on the cells, preferably in a substantial monolayer of carriers disposed over a layer of cells, to define localized subsets 136 of the cells that are apposed to the carriers. Preferably, one or more cells of each subset are transfected by the transfection materials.

FIGS. 5 and 6 show plan and sectional views, respectively, of apposed position 134. Cell subset 136 may be provided by cells disposed between carrier 128 and substrate 124, by cells that overlap the carrier or that the carrier overlies, by cells that contact the carrier, and/or by cells that are within a selected distance from the carrier. The selected distance may be within a fractional distance from the carrier; closest to one of the carriers than the other carriers; and/or a number of cell diameters (for example, one or two) from the carrier surface and/or perimeter, among others. Such a larger area or volume of apposition may include cells that received transfection material by diffusion, to accommodate slight carrier movement, and/or the like. Apposed cells may be connected to the carrier, for example, when the apposed cells are disposed between the carrier and substrate 124.

Carriers may define apposed cell subsets at any suitable time period during a transfection experiment. In some embodiments, carriers are placed on the cells and then left in position until the completion of the transfection analysis. This static positioning thus identifies cell subsets that are in close proximity to the carriers during the analysis. However, in other embodiments, carriers may be moved before the analysis is completed. For example, transfected cell subsets may be identified and positionally defined based on reading the carrier code and defining a corresponding carrier position, for example, a position relative to substrate 124. Once each cell subset has been positionally defined, carriers may be moved or removed, because the position and identity of the subset has been defined relative to the substrate. However, in some cases, cells may connect tightly to the coded carriers, so that any carrier movement brings along connected cells, thereby detaching the cells from the underlying substrate.

### EXAMPLE 4

Combination of Reverse and Forward Transfection Using Coded Carriers

This example describes combining both forward and reverse transfection using distinct surfaces of each carrier; see FIG. 7.

Carriers 128 may be apposed to different subsets of cells on different surfaces 140, 142 of the carriers to achieve combined forward and reverse transfection. Bottom surface 140 of the carrier may provide transfection material to forward transfect a first apposed subset 136, which was attached previously to substrate 124, as described above in Example 3. Opposing or top surface 142 may provide transfection material to reverse transfect a second apposed subset 144. Second apposed subset 144 may be provided by additional cells 146, which may be placed on top of carrier 128, generally in a step separate from disposing cells 122 on substrate 124. Either apposed subset may be connected to carrier 128 or may be unattached. Cells 122 and additional cells 146 may include the same or different cell types, among others. Furthermore, the transfection material connected to surfaces 140, 142 may be the same or different.

The use of opposing surfaces 140, 142 for apposition to cells may be advantageous. The number of cells transfected by each carrier may be increased, generally up to about two-fold per carrier. In addition, with relatively thin carriers and appropriate optics (e.g., large depth of field), cells on the opposing surfaces may be imaged at the same time. Alternatively, with relatively thick carriers and appropriate optics (e.g., small depth of field), cells on the opposing surfaces may be imaged separately, in different focal planes.

One or both of opposing surfaces 140, 142 may include surface relief, as described above in Section I. Accordingly, forward transfection and/or reverse transfection may be conducted with carrier surfaces 140 and/or 142 that are nonplanar, for example, including one or more recesses or projections, such as bumps, ridges, and/or grooves. Such recesses or projections may define compartments in cooperation with the supporting substrate. The compartments may improve access of fluid and reagents to the cells, may decrease surface tension, may improve cell retention, may increase carrier surface area, may promote connection of cells to the carrier surface, and/or the like.

### EXAMPLE 5

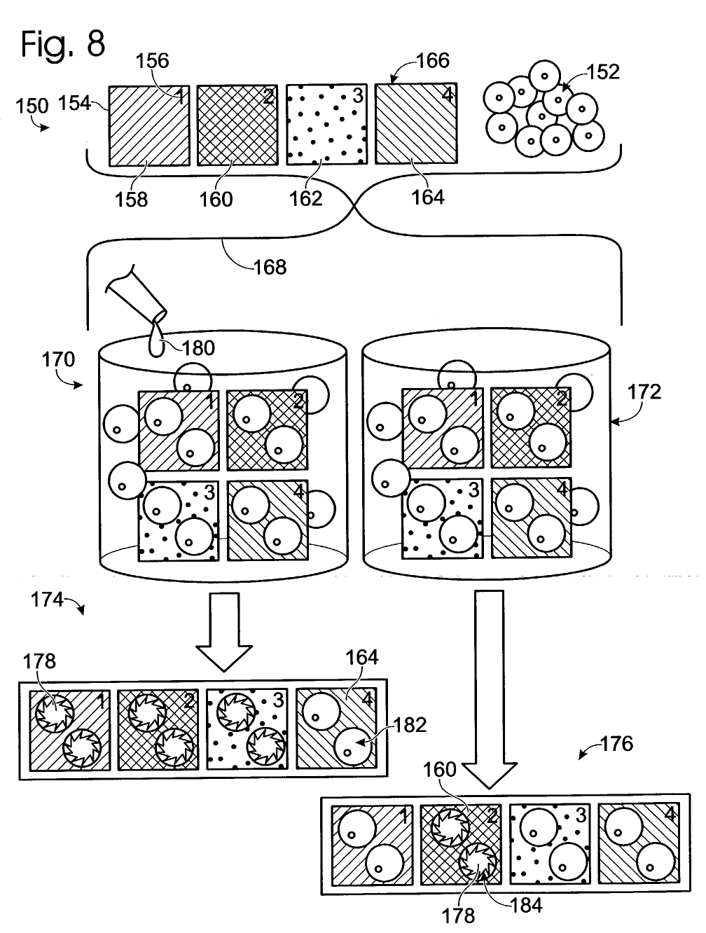

Transfection with Arrays of Antisense Nucleic Acids This example describes a method 150 for transfection of antisense nucleic acids, to identify genes that regulate cellular activities; see FIG. 8.

In method 150, reporter cells 152 may be selected that are easily assayed for a physiological parameter of choice. Here, reporter cells 152 are a stable reporter cell line engineered to express a reporter gene when a signaling cascade is activated. Specifically, reporter cells 152 express GFP when the NF-kappaB cascade is activated. Separate classes of coded carriers 154, each having a distinct code 156, may be connected to different expression vectors 158, 160, 162, 164 to form coded vectors 166. Some or all of the expression vectors may express antisense RNA that specifically binds to RNA expressed from a gene of interest. In alternative embodiments, other types of nucleic acids configured to decrease expression of preselected target genes may be used, such as synthetic antisense nucleic acids, RNAi, etc.

Reporter cells 152 may be apposed to coded vectors 166, shown at 168, to form mixtures 170, 172 of coded antisense vectors and apposed cells. The apposed cells may be transfected with the expression vectors, for example, by forward and/or reverse transfection, as described above. After transfection, each expression vector may express an antisense RNA, which may decrease or abolish expression of the corresponding target gene. As shown at 174 and 176, transfected cells may be assayed for GFP expression. Detectable or increased GFP expression is indicated as a star 178 inside the cells.

Cells measured at 174 are derived from mixture 170 treated with an activator 180 of the NV-kappaB pathway. Since most genes are not expected to be required for the NF-kappaB pathway, most transfected subsets show GFP expression. However, carriers apposed to cell subsets that do not express GFP, such as subset 182, identify antisense nucleic acid 164 and thus may identify a corresponding gene for a positive regulator that was targeted by this antisense nucleic acid. Expression of the positive regulator may be required for activation or functioning of the NF-kappaB pathway.

Negative regulators of the NF-kappaB pathway may be identified using mixture 172 that has not been treated with an NF-kappaB pathway activator, such that the NF-kappaB pathway is generally not active. Since most genes are not expected to encode negative regulators of the NF-kappaB pathway, most transfected cells show no GFP signal, exemplified in carriers with codes “1”, “3”, and “4,” as shown at 176. However, GFP signal 178 is detectable in cell subset 184 that was transfected with antisense expression vector 160. This suggests that a corresponding antisense-targeted gene is a negative regulator of the NF-kappaB pathway, since decreased levels of target gene expression resulted in activation of the pathway.

The multiplexed analysis may be modified to measure more than one physiological parameter. For example, a different nucleic acid configured to decrease expression of a gene of interest may be connected to different classes of coded carriers, and a different reporter cell line that measures activity of a distinct cell pathway may be apposed to each different carrier class. Transfection of each distinct reporter cell line with the nucleic acid may decrease expression of the gene of interest. Any role of the gene of interest in each distinct cell pathway may be measured by a change in activity of the corresponding reporter cell line. For example, reporter cell lines may be used that express GFP in response to the activation of different signaling cascades, such as NF-kappaB, CREB, Gs-GPCR, NFAT, ELK, MAP kinase, or the like. After transfection of an antisense nucleic acid or antisense vector into the reporter cell lines, the cell lines may be analyzed for GFP expression, either in the presence or absence of pathway activators, to determine if the gene of interest is involved in regulation, either positive or negative, of these signaling pathways. This method measures the role of a single gene on multiple physiological readouts. In alternative embodiments, the reporter cell lines may be connected to coded carriers first and then transfected with transfection materials. In other embodiments, more than one nucleic acid may be transfected into more than one reporter cell line. These embodiments multiplex-analyze the roles of more than one gene on more than one pathway. For example, reporter cell lines for the signaling pathways mentioned above each may be transfected in apposition to more than one different class of coded carrier with different nucleic acids for decreasing expression.

### EXAMPLE 6

Transfection with Coded Arrays of Sense Nucleic Acids

This example describes multiplexed assays performed by transfection of sense nucleic acids encoding regulators.

In other multiplexed cell transfection assays, information about a cell population (or individual cells within the population) may be determined by transfection of a sense expression vector or nucleic acid, by forward and/or reverse transfection. The sense expression vector or nucleic acid may encode a wild type, mutant, or fusion derivative of a protein of interest, particularly a regulator. These transfections of sense nucleic acids (or sense-encoding nucleic acids) may be used to identify cells that do not respond to the regulator. Cells may not respond for various reasons, generally due to changes in the structure (i.e., mutation or modification, among others) or expression level of a target of the regulator. For example, the cells may express a nonbinding derivative of the target or may overexpress the target, when the target is a protein. Similarly, when the target is nucleic acid, the target may be amplified or mutated so that overexpression of the transfected regulator has little or no effect.

### EXAMPLE 7

Carrier Treatments

This example describes carrier treatments that may stabilize interaction between transfection materials and carriers, may more effectively connect transfection materials to carriers, and/or may limit diffusion of transfection materials. The treatments may be used to produce a more localized transfection on a portion of a carrier and/or to expose cells to a higher concentration of transfection materials, among others. Furthermore, the treatment may obviate any need for gelatin when connecting transfection materials to carriers.

Carriers may be treated to modify their surface chemistry. Glass carriers may be exposed to polylysine, an extracellular component or mixture, and/or an extracellular matrix extract, such as MATRIGEL. Alternatively, or in addition, glass carriers may be exposed to 2% aminosilane in acetone for one hour, and then briefly washed with 0.3 M sucrose/phosphate-buffered saline (PBS) after coating. Solutions of transfection materials then may be applied to the carriers to connect the transfection materials. In some embodiments, gelatin may not be included with the transfection materials.

### EXAMPLE 8

Transfection Using Dendrimer Transfection Reagent

This example describes the use of a dendrimer reagent that may reduce diffusion of transfection materials and/or restrict movement of cells away from the carriers to which they are connected.

The “diffusion effect” describes a situation in which transfected cells are located at a distance from carriers. This effect may have one or more causes. For example, transfection material may dissociate from a carrier and transfect cells at a location that is spaced away from the carrier. Alternatively, or in addition, cells may detach from carriers after being transfected, and then move (or be moved) away from the carrier. Furthermore, carriers may move relative to the cell substrate, leaving transfected cells behind. Whatever the cause, the diffusion effect may contribute to misidentification of “stray” positive cells, because they appear to be apposed to other carriers. This may reduce signal and increase noise, decreasing data accuracy and potentially leading to false positives in screening assays.

An activated dendrimer-type transfection reagent may be used in transfections with carriers to reduce the diffusion effect. A suitable dendrimer reagent may be cationic, such as the SUPERFECT transfection reagent from QIAGEN. The dendrimer reagent may be used instead of, or in addition to, a cationic lipid in transfections. The dendrimer may reduce the diffusion effect because the dendrimer may form a dendrimer/DNA complex that interacts more strongly with the carrier surface than a corresponding lipid/DNA complex, thus more effectively immobilizing the DNA. Alternatively, the dendrimer may speed connection of cells to carriers or strengthen cell adhesion, thereby keeping transfected cells localized.

The dendrimer reagent may be combined with carriers and transfection materials at any suitable time. For example, the dendrimer reagent may be combined with carriers before, after, or concurrently with connection of the transfection materials to the carriers. In some embodiments, the dendrimer reagent (with or without transfection material) may be combined with glass carriers that have been pretreated with polylysine.

### EXAMPLE 9

Transfection using Coded Carriers Formed from PEMA

This example describes the use of plastic-based carriers for transfection. The plastic carriers may be constructed of any suitable plastic, such as acrylates. An exemplary acrylate is poly(ethylmethacrylate), abbreviated as PEMA. PEMA carriers may be formed to include a code by any suitable method, such as laminating colored layers, stamping, printing, etc.

PEMA coded carriers may be treated with any suitable materials to modify the surface chemistry or characteristics of the carriers for transfection, or may be left untreated. Suitable materials may react covalently or bind noncovalently to the carrier surface or interior. Such materials may include allyl amine, ammonia, or carbon dioxide, among others. Alternatively, or in addition, such materials may be a transfection reagent(s) and/or adhesive(s) that is combined with the carriers before, during, and/or after transfection materials are connected to the carriers. For example, PEMA carriers may be pre-coated with Matrigel or any other such matrix before addition of DNA/transfection reagent to increase transfection efficiency. In general, PEMA carriers may be more amenable to surface derivatization than glass carriers for the enhancement of nucleic acid, lipid, dendrimer, protein or other molecular interactions with the carrier surface.

Cationic lipids or activated dendrimer, among others, have been used successfully. as transfection reagents with PEMA carriers. Transfection efficiencies using PEMA carriers with either transfection reagent have been equivalent to or greater than those using glass carriers. As with glass carriers, dendrimer may reduce the “diffusion effect” compared to lipid when PEMA carriers are used.

### EXAMPLE 10

Exemplary Experiments using Coded Carriers for Multiplexed Analysis of Transfected Receptors

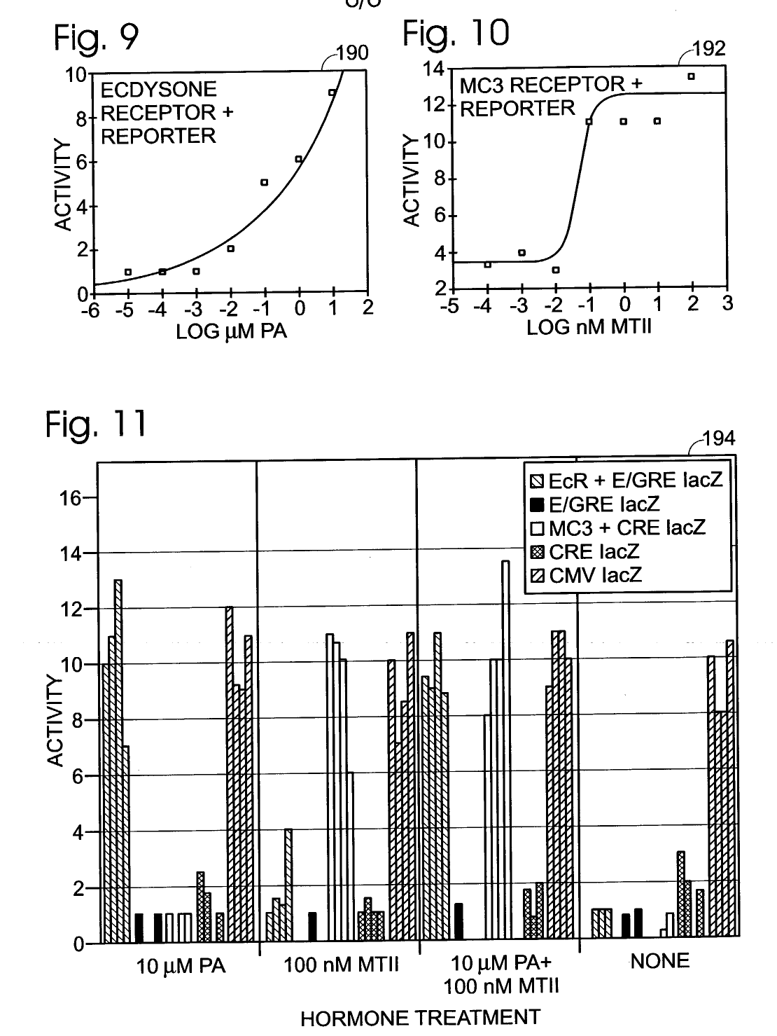

This example describes experimental results obtained using coded carriers to transfect GPCRs and nuclear receptors into cells; see FIGS. 9-11.

FIGS. 9 and 10 show dose-response curves produced by transfecting receptor expression vectors and corresponding reporter genes into cells. For each set of experiments, the indicated vector and reporter gene were connected to coded carriers, apposed to HEK 293 cells, and then transfected into subsets of the cells.

FIG. 9 is a graph 190 of dose-response data for an ecdysone response system. An ecdysone receptor expression vector (EcR) and an ecdysone-responsive reporter gene (E/GRE lacZ) were transfected into cells using coded carriers placed in different wells. The cells were then treated in the different wells with the indicated concentrations of ponasterone (PA), an agonist for the ecdysone receptor. The amount of expressed beta-galactosidase activity was then measured and plotted.

FIG. 10 is a graph 192 of dose-response data for response of a transfected MC3 receptor. An MC3 receptor expression vector (MC3) and an MC3 receptor-responsive reporter gene (CRE lacZ) were transfected into cells using coded carriers placed in different wells. The cells were then treated in the different wells with the indicated concentrations of MTII, a synthetic agonist for the MC3 receptor. The amount of expressed beta-galactosidase actvity was then measured and plotted. In each case, the dose-response obtained by transfection using coded carriers was similar to that obtained by standards methods.

FIG. 11 is a bar graph 194 showing selective agonist stimulation of ecdysone and MC3 response systems introduced by multiplexed transfection with coded carriers. The indicated combinations of nucleic acids (reporter gene alone, or reporter gene and receptor expression vector) were connected to different classes of coded carriers, mixed, and then placed in a plurality of microplate wells. HEK 293 cells were apposed to the carriers in each well, and then the nucleic acids transfected into apposed subsets of the cells. Different wells received different agonists or agonist mixtures, as indicated along the abscissa. Beta-galactosidase activity for the subsets was measured and related to the corresponding transfected nucleic acid by reading the codes of the carriers. The beta-galactosidase activity was plotted on the ordinate based on the hormone(s) added and the nucleic acid(s) transfected.

The results presented in graph 194 indicate the expected specificity of hormone response. The transfected ecdysone responsive reporter gene (E/GRE lacZ) was activated substantially only upon co-transfection with the ecdysone receptor and in the presence of PA alone, or PA plus MTII. Similarly, the transfected MC3-responsive reporter (CRE lacZ) was activated substantially only upon co-transfection with the MC3 receptor and in the presence of MTII alone, or MTII plus PA. The reporter CMV lacZ is a positive control that is active under all conditions tested.

### EXAMPLE 11

Selected Embodiments 1

This example describes additional selected embodiments of the invention, presented as a series of indexed paragraphs.

1. A method of transfecting cells, comprising (1) associating, for a set of microcarriers, each microcarrier with a transfection material, the transfection material associated with at least two microcarriers being different, each microcarrier including a code that identifies the associated transfection material; (2) associating a cell population with each microcarrier; (3) transfecting the cell population with the associated transfection material; and (4) reading the code of at least one microcarrier to identify the transfection material that transfected the cell population associated with such microcarrier.

2. The method of paragraph 1, wherein the cell population is associated after the transfection material is associated.

3. The method of paragraph 1, wherein the set includes at least three different codes, each of the different codes identifying a different associated transfection material.

4. The method of paragraph 1, wherein the transfection material includes one or more nucleic acids.

5. The method of paragraph 4, wherein the transfection material is selected from the group consisting of antisense nucleic acids, expression vectors, reporter gene vectors, synthetic oligonucleotides, post-transcriptional gene silencing agents, double-stranded RNA, and RNAi.

6. The method of paragraph 4, wherein the transfection material encodes an effector and a reporter gene.

7. The method of paragraph 4, wherein the transfection material includes an antisense nucleic acid, antisense expression vector, or PTGS agent configured to decrease expression of a preselected target gene.

8. The method of paragraph 1, wherein the set of microcarriers is associated with the different transfection materials separately.

9. The method of paragraph 8, further comprising the step of mixing the separately associated transfection materials so that such transfection materials are distributed arbitrarily.

10. The method of paragraph 1, wherein the microcarriers at least substantially includes at least one of glass and poly(ethylmethacrylate).

11. The method of paragraph 1, wherein the microcarriers are treated to modify a surface of the microcarriers.

12. The method of paragraph 11, wherein the microcarriers are treated with a material selected from the group consisting of aminosilanes, polylysine, dendrimers, atelocollagen, and an extracellular matrix extract.

13. The method of paragraph 11, wherein the microcarriers are not treated with gelatin.

14. The method of paragraph 1, wherein the code is optically detectable.

15. The method of paragraph 14, wherein the code relies at least partially on detectable colors.

16. The method of paragraph 1, further comprising measuring a characteristic of the cell population associated with the at least one microcarrier.

17. The method of paragraph 16, wherein the characteristic is measured as a combined signal from plural cells of the cell population.

18. The method of paragraph 16, wherein the characteristic relates to a signal measured from an individual cell.

19. The method of paragraph 1, wherein the transfection material is selected from the group consisting of proteins, viruses, carbohydrates, chemical modulators and portions thereof.

20. The method of paragraph 1,wherein the step of associating the transfection material includes treating at least some microcarriers of the set with an adhesion promoter configured to increase association of such microcarriers with their corresponding transfection materials.

21. The method of paragraph 20, wherein the adhesion promoter is selected based on the associated transfection material, the adhesion promoter being different for some microcarriers of the set.

22. The method of paragraph 20, wherein the adhesion promoter is attached to microcarriers of the set through noncovalent interactions.

23. The method of paragraph 20, wherein the adhesion promoter is selected from the group consisting of macromolecules that binds nucleic acid, compounds that bind nucleic acids, ethidium monoazide, and nucleic acids.

24. The method of paragraph 1, wherein the step of associating the transfection material includes combining the transfection material with at least one transfection vehicle.

25. The method of paragraph 24, wherein the at least one transfection vehicle is selected from the group consisting of a precipitate, a vesicle, a micelle, a liposome, a virus, a peptide carrier, atelocollagen, polyethylenimine, a dendrimer transfecton reagent, and polyethylene glycol.

26. The method of paragraph 24, wherein the at least one transfection vehicle is selected based on the transfection material, the at least one transfection vehicle being different for some microcarriers of the set.

27. The method of paragraph 1, wherein at least a subset of the population of cells produces a releasing activity that releases the transfection material from association with the carrier.

28. The method of paragraph 27, wherein the releasing activity is an enzyme, the enzyme being reactive with at least one of the transfection material, an adhesion promoter that facilitates the step of associating the transfection material, and a bridging moiety disposed between the transfection material and its associated carrier.

29. The method of paragraph 1, wherein at least a portion of each cell population is attached to a substrate before the portion is associated with its corresponding microcarrier, the substrate being distinct from the set of microcarriers.

30. The method of paragraph 29, the substrate being a well of a microplate.

31. The method of paragraph 29, the portion being a first portion and being associated with a first surface of the carrier, a second portion of the cell population being separate from the substrate and being associated with a second surface of the microcarrier after the first portion is associated.

### EXAMPLE 12

Selected Embodiments 2

This example describes further selected embodiments of the invention, presented as a series of indexed paragraphs.

1. A method of transfecting cells, comprising (1) attaching cells to a substrate; (2) associating, for a set of microcarriers, each microcarrier of the set with a transfection material to form a transfection mixture, the set of microcarriers being distinct from the substrate, the transfection material associated with at least two microcarriers of the set being different, each microcarrier of the transfection mixture including a code that identifies the associated transfection material; (3) contacting the attached cells with the transfection mixture so that a subset of the cells is transfected with the transfection material associated with at least one of the microcarriers; and (4) reading the code of the at least one microcarrier to identify the associated transfection material, thereby relating such transfection material to the subset of cells.

2. The method of paragraph 1, the substrate and each microcarrier of the set having a length, the length of the substrate being substantially greater than the length of each microcarrier.

3. The method of paragraph 1, the substrate being defined by a well of a microplate.

4. The method of paragraph 1, the cells being a substantially homogeneous population.

5. The method of paragraph 1, the transfection material being at least one nucleic acid.

6. The method of paragraph 1, the transfection material including a reporter gene.

7. The method of paragraph 1, the transfection material being configured to express a test protein whose activity is being measured.

8. The method of paragraph 1, wherein the transfection material includes a nucleic acid configured to decrease expression of a preselected target gene.

9. The method of paragraph 1, the subset remaining at least substantially attached to the substrate.

10. The method of paragraph 1, the subset becoming at least partially attached to the at least one microcarrier before the step of reading.

11. The method of paragraph 1, the step of contacting including arbitrarily disposing the microcarriers of the transfection mixture to positions on the substrate.

12. The method of paragraph 1, wherein the step of contacting apposes a surface of the at least one microcarrier with a surface of the substrate to define an area of the substrate to which the subset of cells is attached.

13. The method of paragraph 12, the area being larger than the surface of the at least one microcarrier.

14. The method of paragraph 12, wherein the area has a position within the substrate, and the step of reading includes measuring the position.

15. The method of paragraph 1, further comprising moving the at least one microcarrier relative to the substrate and measuring a characteristic of the transfected subset after the step of moving.

16. The method of paragraph 1, the method further comprising the step of attaching other cells to the at least one microcarrier so that the transfection material transfects the other cells.

17. The method of paragraph 16, the at least one microcarrier having opposing surfaces, the subset of cells and the other cells being attached respectively to different opposing surfaces.

18. The method of paragraph 1, the method further comprising the step of measuring a characteristic of the transfected subset of cells.

Example 13. Selected Embodiments 3

This example describes further selected embodiments of the invention, presented as a series of indexed paragraphs.

1. A method of transfecting cells, comprising: (1) connecting cells to a substrate; (2) placing a mixture of coded carriers adjacent the substrate to appose the cells to the mixture, the mixture including at least two carrier classes, each carrier class being connected to a different transfection material and having a code that identifies the different transfection material; and (3) introducing each different transfection material into at least one of the cells from the carrier class to which the different transfection material is connected; and

reading the code of at least one carrier class to identify the different transfection material introduced into the at least one cell.

2. The method of paragraph 1, wherein the step of placing includes mixing the at least two carrier classes to form the mixture and then apposing the cells to the mixture.

3. The method of paragraph 1, wherein each of the different transfection materials includes one or more nucleic acids.

4. The method of paragraph 3, wherein the one or more nucleic acids are configured to at least one of decrease expression of a preselected target gene, introduce a mutation, act as a reporter gene, and express a protein of interest.

5. The method of paragraph 1, wherein each at least one cell is a plurality of different subsets of the cells, and wherein each carrier class includes a plurality of the coded carriers disposed in individual apposition to the plurality of different subsets.

6. The method of paragraph 1, wherein the step of placing includes connecting at least a portion of the cells to the carriers so that the portion is connected to both the substrate and the mixture.

7. The method of paragraph 1, wherein the substrate is provided by a well of a microplate.

8. The method of paragraph 1, wherein a subset of the cells is disposed between each carrier class and the substrate, the at least one cell being included in the subset.

9. The method of paragraph 1, wherein the at least one cell is more closely apposed to one coded carrier of the carrier class to which the different transfection material is connected than to other coded carriers of the mixture.

10. The method of paragraph 1, wherein the steps of connecting, placing, and introducing are repeated a plurality of times in a plurality of wells of a microplate.

11. The method of paragraph 1, wherein the step of placing at least substantially covers the cells and the mixture of coded carriers with fluid in a shared compartment over the substrate.

12. The method of paragraph 1, wherein the cells are in numerical excess over the carriers.

13. The method of paragraph 1, wherein the substrate has a maximum linear dimension and the carriers have an average length, the maximum linear dimension being at least about two-fold greater than the length.

14. The method of paragraph 1, wherein the coded carriers each have a first and a second opposing surface, the step of placing apposing the cells to the first surface of at least a subset of carriers, the method further comprising the step of placing other cells on the second surface of the subset.

15. A method of transfecting cells, comprising: (1) connecting transfection materials to at least two classes of carriers, each class being connected to a different transfection material and having an optically detectable code that identifies the different transfection material; (2) mixing the classes to randomly position the carriers relative to one another in a mixture; (3) connecting cells to a substrate; (4) placing the mixture adjacent the substrate after connecting the cells to appose the cells to the carriers; (5) introducing each different transfection material into at least one of the cells from the carrier class to which the different transfection material is connected; and (6) reading the code of at least one carrier class to identify the different transfection material introduced into the at least one cell.

16. The method of paragraph 15, wherein the step of placing connects at least a portion of the cells to the carriers.

17. The method of paragraph 15, wherein the substrate is one of a plurality of substrates defined by wells of a microplate, and wherein the steps of connecting, placing, and introducing are performed a plurality of times in the wells.

18. The method of paragraph 15, wherein each transfection material includes one or more nucleic acids, the one or more nucleic acids being configured to at least one of decrease expression of a preselected target gene, introduce a mutation, act as a reporter gene, and express a protein of interest.

19. The method of paragraph 15, wherein a subset of the cells is disposed between each carrier class and the substrate, the at least one cell being included in the subset.

20. A method of screening with cells, comprising: (1) placing carriers of at least two classes at random relative positions within a compartment adjacent a substrate and in apposition to cells that are connected to the substrate, each carrier class being connected to a different transfection material and having a code that identifies the different transfection material; (2) introducing each different transfection material into at least one of the cells from the carrier class to which the different transfection material is connected; (3) exposing the cells to a candidate modulator; (4) measuring an effect of the candidate modulator on each at least one cell; and (5) reading the code of at least one carrier class to identify the different transfection material introduced into the at least one cell and to relate the effect of the candidate modulator to the different transfection material introduced into the at least one cell.

21. The method of paragraph 20, wherein the steps of placing, introducing, exposing, and measuring are repeated a plurality of times in different compartments with different candidate modulators.

22. The method of paragraph 21, wherein the different compartments are different wells of a microplate.

23. The method of paragraph 20, wherein the effect is selected from the group consisting of no detectable effect and detectable effects.

24. The method of paragraph 20, wherein the at least one cell is more closely apposed to one coded carrier of the carrier class to which the different transfection material is connected than to other coded carriers of the mixture.

25. The method of paragraph 20, wherein the coded carriers each have a first and a second opposing surface, the step of placing apposing the cells to the first surface of at least a subset of carriers, the method further comprising the step of placing other cells on the second surface of the subset.

26. The method of paragraph 25, wherein the step of introducing also introduces each different transfection material into at least one of the other cells from the carrier class to which the different transfection material is connected.

27. A system for screening transfected cells with modulators, comprising: (1) means for placing carriers of at least two classes at random relative positions within a compartment adjacent a substrate and in apposition to cells that are connected to the substrate, each carrier class being connected to a different transfection material and having a code that identifies the different transfection material; (2) means for introducing each different transfection material into at least one of the cells from the carrier class to which the different transfection material is connected; (3) means for exposing the cells to a candidate modulator; (4) means for measuring an effect of the candidate modulator on each at least one cell; and (5) means for reading the code of at least one carrier class to identify the different transfection material introduced into the at least one cell and to relate the effect of the candidate modulator to the different transfection material introduced into the at least one cell.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of transfecting cells, comprising:
providing cells apposed to a mixture of coded carriers, the mixture including at least two carrier classes, each carrier class being connected to a different transfection material and having a code that identifies the different transfection material;
introducing each different transfection material into at least one of the cells from the carrier class to which the different transfection material is connected; and
reading the code of at least one carrier class to identify the different transfection material introduced into the at least one cell.

2. The method of claim 1, wherein the step of providing includes mixing the at least two carrier classes to form the mixture and then apposing the cells to the mixture.

3. The method of claim 1, wherein each of the different transfection materials includes one or more nucleic acids.

4. The method of claim 3, wherein the one or more nucleic acids are configured to at least one of decrease expression of a preselected target gene, introduce a mutation, act as a reporter gene, and express a protein of interest.

5. The method of claim 1, wherein each at least one cell is a plurality of different subsets of the cells, and wherein each carrier class includes a plurality of the coded carriers disposed in individual apposition to the plurality of different subsets.

6. The method of claim 1, wherein the step of providing includes connecting at least a portion of the cells to the carriers.

7. The method of claim 1, wherein the step of providing includes placing the carriers on a substrate, at least a portion of the cells being connected to the substrate before placing.

8. The method of claim 1, wherein a subset of the cells is apposed to each carrier class, the at least one cell corresponding to less that than all of the subset.

9. The method of claim 1, wherein the at least one cell is more closely apposed to one coded carrier of the carrier class to which the different transfection material is connected than to other coded carriers of the mixture.

10. The method of claim 1, wherein the step of providing is repeated a plurality of times in a plurality of wells of a microplate.

11. The method of claim 1, wherein the step of providing includes placing the cells and the mixture of coded carriers together in fluid within a compartment.

12. The method of claim 1, wherein the cells are in numerical excess over the carriers.

13. A method of transfecting cells, comprising:
connecting transfection materials to at least two classes of carriers, each class being connected to a different transfection material and having an optically detectable code that identifies the different transfection material;
mixing the classes to randomly position the carriers relative to one another;
apposing cells to the carriers;
introducing each different transfection material into at least one of the cells from the carrier class to which the different transfection material is connected; and
reading the code of at least one carrier class to identify the different transfection material introduced into the at least one cell.

14. The method of claim 13, wherein the step of apposing connects at least a portion of the cells to the carriers.

15. The method of claim 13, wherein the step of apposing is conducted after the step of mixing.

16. The method of claim 13, the step of apposing including connecting at least a portion of the cells to a substrate that is distinct from the carriers and placing the coded carriers adjacent the substrate, wherein the step of connecting the portion of the cells is performed before the step of placing.

17. The method of claim 13, wherein each transfection material includes one or more nucleic acids, the one or more nucleic acids being configured to at least one of decrease expression of a preselected target gene, act as a reporter gene, and express a protein of interest.

18. The method of claim 13, the carriers being disposed in a plurality of discrete compartments, wherein the step of introducing is conducted at least substantially in each of the discrete compartments.

19. The method of claim 13, wherein the at least one cell is more closely apposed to one coded carrier of the carrier class to which the different transfection material is connected than to other coded carriers of the mixture.

20. A method of screening with cells, comprising:
placing carriers of at least two classes at random relative positions within a compartment and in apposition to cells, each carrier class being connected to a different transfection material and having a code that identifies the different transfection material;
introducing each different transfection material into at least one of the cells from the carrier class to which the different transfection material is connected;
exposing the cells to a candidate modulator;
measuring an effect of the candidate modulator on each at least one cell; and
reading the code of at least one carrier class to identify the different transfection material introduced into the at least one cell and to relate the effect of the candidate modulator to the different transfection material introduced into the at least one cell.

21. The method of claim 20, wherein the steps of introducing, exposing, and measuring are repeated a plurality of times in different compartments with different candidate modulators.

22. The method of claim 21, wherein the different compartments are different wells of a microplate.

23. The method of claim 20, wherein the effect is selected from the group consisting of no detectable effect and detectable effects.

24. The method of claim 20, wherein the at least one cell is more closely apposed to one coded carrier of the carrier class to which the different transfection material is connected than to other coded carriers of the mixture.

25. A kit for transfection of cells, comprising:
a set of at least two classes of microcarriers, each class being connected noncovalently to a different transfection material and having an optical code that identifies the different transfection material, the transfection materials being configured to be at least partially released when incubated with cells in an aqueous environment.

26. The kit of claim 25, wherein the at least two classes include at least three classes of microcarriers.

27. A composition for multiplexed cell analysis, comprising:
a mixture of coded carriers, the mixture including at least two carrier classes, each carrier class being connected to a different transfection material and having a code that identifies the different transfection material; and
cells apposed to the mixture.

28. A system for screening transfected cells with modulators, comprising:
means for placing carriers of at least two classes at random relative positions within a compartment and in apposition to cells, each carrier class being connected to a different transfection material and having a code that identifies the different transfection material;
means for introducing each different transfection material into at least one of the cells from the carrier class to which the different transfection material is connected;
means for exposing the cells to a candidate modulator;
means for measuring an effect of the candidate modulator on each of the at least one cell; and
means for reading the code of at least one carrier class to identify the different transfection material introduced into the at least one cell and to relate the effect of the candidate modulator to the different transfection material introduced into the at least one cell.
